(19) United States
(12) Patent Application Publication
(10) Pub. No.: US 2024/0140913 A1
(43) Pub. Date: May 2, 2024

(54) A PROCESS FOR PREPARING ABEDITEROL AND INTERMEDIATES THEREOF

(71) Applicant: GBR LABORATORIES PRIVATE LIMITED, HYDERABAD (IN)

(72) Inventors: Pratap Reddy Gaddam, Hyderabad (IN); Samhitha Reddy Gaddam, Hyderabad (IN); Madaalasa Reddy Gaddam, Hyderabad (IN); Udaya Kumar Reddy Mosali, Hyderabad (IN)

(73) Assignee: GBR LABORATORIES PRIVATE LIMITED, HYDERABAD (IN)

(21) Appl. No.: 18/276,135

(22) PCT Filed: Feb. 9, 2022
(86) PCT No.: PCT/IN2022/050112

§ 371 Date: Aug. 7, 2023

(30) Foreign Application Priority Data
Feb. 9, 2021 (IN) 202141005529

(51) Int. Cl. C07D 215/26

(52) U.S. Cl. CPC C07D 215/26

(57) ABSTRACT

The present invention relates to a process for preparation of Abediterol of the Formula (I) or its pharmaceutically acceptable salts, and intermediate compounds including the compound of the Formula (VII) by chiral sulfide mediated epoxidation. The process involves preparation of the intermediate compounds followed by preparation of Abediterol from the intermediate compounds. The compound Abediterol having the formula I is synthesized by various routes from the intermediate compound of Formula (VII). The process is cost effective and gives higher yield and better purity. The process of the present invention uses simple raw materials and reagents; and does not use hypertoxic materials.

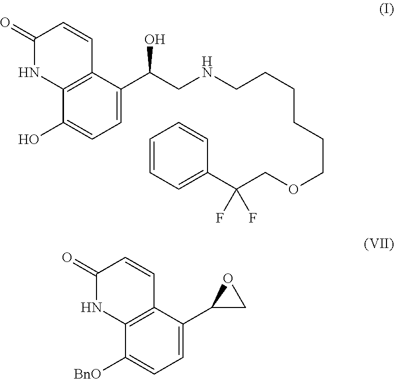 [SMILES: O=c1ccc2c([C@@H](O)CNCCCCCCOCC(F)(F)c3ccccc3)ccc(O)c2[nH]1 ; O=c1ccc2c([C@@H]3CO3)ccc(OCc3ccccc3)c2[nH]1]

### CROSS-REFERENCE TO RELATED APPLICATIONS

This application is a national phase entry of PCT Application PCT/IN2022/050112, filed Feb. 9, 2022 titled “A PROCESS FOR PREPARING ABEDITEROL AND INTERMEDIATES THEREOF” which claims priority to India Patent Application No. 202141005529, filed Feb. 9, 2021, titled “A PROCESS FOR PREPARING ABEDITEROL AND INTERMEDIATES THEREOF”, both of which are incorporated herein in their entirety by reference.

### FIELD OF THE INVENTION

The present invention relates to a process for preparation of chiral intermediates of long-acting β-agonist (LABAs) and more particularly to a process for preparation of chiral intermediates of Abediterol by chiral sulfide mediated epoxidation.

### BACKGROUND OF THE INVENTION

Respiratory disorders are one of the leading causes of death in the world. Respiratory disorders are associated mainly with tobacco smoking, air pollution or occupational exposure, which can cause obstruction of airflow in the lungs resulting in bouts of breathlessness. COPD, bronchial asthma, chronic bronchitis, asthmatic bronchitis and emphysema are some of the respiratory disorders.

Bronchodilators are frequently used to treat respiratory disorders. The bronchodilators help loosen tight muscles of the airways leading to the widening of airways. The widening of airways in turn leads to easy breathing. Various class of compounds work as bronchodilators such as the β-adrenoceptor agonists, muscarinic receptor antagonists and the like. These bronchodilators are available in both short acting and long acting forms.

A variety of β2-adrenoceptor agonists with long half-lives, also called long-acting β2-adrenoceptor agonists (LABAs) are currently under development. LABAs exert their effect by stimulating the intracellular adenylyl cyclase which catalyzes the conversion of adenosine triphosphate (ATP) to cyclic 3′,5′-adenosine monophosphate (cAMP). Increase in cyclic AMP is associated with relaxation of bronchial smooth muscle and inhibition of release of hypersensitivity mediators from mast cells in the lungs. One such LABA is the Abediterol. Abediterol is currently under development as a long-acting bronchodilator. Abediterol displays superior bronchodilatory potency and similar or superior selectivity for β2-adrenoreceptors over β1-adrenoreceptors.

There is a growing interest in the development of cost effective and environmentally friendly processes for β2-adrenoceptor agonists (LABA). There are known processes for the preparation of certain β2-adrenoceptor agonists (LABA) and its intermediates. The research article titled “Synthesis of the Lipophilic Amine Tail of Abediterol Enabled by Multiphase Flow Transformations” by Jorge Garcia-Lacuna et al in the Journal “Organic Process Research & Development” describes a process for preparation of the lipophilic amine tail portion of Abediterol. The process comprises of the steps of a phase-transfer-catalyzed liquid/liquid O-alkylation, a rhodium-catalyzed hydroformylation, and a ruthenium-catalyzed reductive amination. The research article “Optimization and sustainability assessment of a continuous flow Ru-catalyzed ester hydrogenation for an important precursor of a β2-adrenergic receptor agonist” by Michael Prieschl et al in the Journal “Green Chemistry” discloses a process of ruthenium-catalyzed continuous flow ester hydrogenation using hydrogen (H₂) gas for the synthesis of 2,2-Difluoro-2-phenylethanol which is a key precursor of Abediterol.

The compound Abediterol is derived from a class of compounds called Quinolinones. The patent U.S. Pat. No. 7,521,558B2 by Theravance Inc discloses a crystalline form of biphenyl compound, and a process of preparing the compound involving formation of the intermediates 2-Quinolinones. The patent application WO2006122788A1 by Almirall Prodesfarma SA et al describes 4-(2-amino-1-hydroxyethyl) phenol derivatives as β2 adrenergic agonists involving formation of intermediates 2-Quinolinones.

These are some of the known processes for the preparation of some certain long-acting β2-adrenoceptor agonists (LABAs) and their intermediates. However, the known processes are expensive and have extended production time. There is need to for a process of preparation of chiral intermediates of Abediterol which is cost effective, less time consuming and has fewer steps of synthesis. There is a further need of a process that avoids the usage of toxic reagents like borane derivative during the (chiral) selective reduction.

Further, there is a need for an industrially feasible process for preparation of Abediterol and its chiral intermediates with improved yield and purity, thereby reducing production cost and time. Also, there is a need to synthesize Abediterol from simple raw materials using safe and simple process.

### SUMMARY OF THE INVENTION

The present invention describes a process for preparation of Abediterol compound having the Formula I or pharmaceutically acceptable salts, and the process for the preparation of chiral intermediate compounds of Formula I.

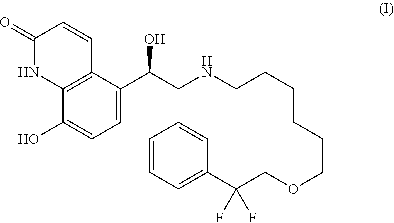 [SMILES: O=c1ccc2c([C@@H](O)CNCCCCCCOCC(F)(F)c3ccccc3)ccc(O)c2[nH]1]

The process includes the steps of addition of the compound of Formula (II) to a solvent followed by adding a base and a benzylating agent to obtain a compound of Formula (III); followed by addition of the compound of Formula (III) to an acid and a brominating agent to make a reaction mixture; to obtain a compound of Formula (IV); carbonylation of compound of formula IV with strong base to give compound of formula V; chiral epoxidation of compound of formula V using chiral sulphide (VI), in presence of base to give compound of formula VII; followed by synthesis of Abediterol having the Formula (I) from the compound of Formula (VII) via intermediates of the Formula (X) and Formula (XI); or
(ii) via intermediates of the Formula (IX) and Formula (XI); or
(iii) via intermediates of the Formula (X) and Formula (XIV); or
(iv) via intermediates of the Formula (IX) and Formula (XIV); or
(v) via intermediates of the Formula (IXa) and Formula (XVI); or
(vi) via intermediates of the Formula (XVIII) and Formula (XVI); or
(vii) via intermediates of the Formula (XX) and Formula (XVI).

The process of synthesis of Abediterol from intermediate (VII) via intermediates of the Formula (X) and Formula (XI) includes the steps of cleavage of chiral epoxide of compound of formula VII using brominating agent to give compound of formula VIII; protection of compound of formula VIII with suitable protecting agent to give compound of formula IX; debenzylation of compound of formula IX using debenzylating agent to give compound of formula X; condensation of compound of formula X with formula XI in presence of base to give compound of formula XII; and deprotection of compound of formula XII using deprotecting agent to give compound of the Formula I.

The process of synthesis of Abediterol from intermediate (VII) via intermediates of the Formula (IX) and Formula (XI) includes the steps of cleavage of chiral epoxide compound of formula VII using brominating agent to give compound of formula VIII; protection of compound of formula VIII with suitable protecting agent to give compound of formula IX; condensation of compound of formula IX with formula XI in presence of base to give compound of formula XIII; and optionally using debenzylation or deprotection of compound of formula XIII in presence of debenzylating agent or deprotecting agent.

The process of synthesis of Abediterol from intermediate (VII) via intermediates of the Formula (X) and Formula (XIV) includes the steps of cleavage of chiral epoxide compound of formula VII using brominating agent to give compound of formula VIII; protection of compound of formula VIII with suitable protecting agent to give compound of formula IX; debenzylation of compound of formula IX using debenzylating agent to give compound of formula X; condensation of compound of formula X with formula XIV in presence of base.

The process of synthesis of Abediterol from intermediate (VII) via intermediates of the Formula (IX) and Formula (XIV) includes the steps of cleavage of chiral epoxide compound of formula VII using brominating agent to give compound of formula VIII; protection of compound of formula VIII with suitable protecting agent to give compound of formula IX; debenzylation of compound of formula IX using debenzylating agent to give compound of formula X; condensation of compound of formula X with formula XIV in presence of base.

The process of synthesis of Abediterol from intermediate (VII) via intermediates of the Formula (IXa) and Formula (XVI) includes the steps of cleavage of chiral epoxide compound of formula VII using suitable amine derivative to give compound of formula VIII (a); protection of compound of formula VIII (a) with suitable protecting agent to give compound of formula IX (a); condensation of compound of formula IX (a) with formula XVI in presence of base to give compound of formula XVII; and optionally using debenzylation or deprotection of formula XVII in presence of debenzylating agent or deprotecting agent.

The process of synthesis of Abediterol from intermediate (VII) via intermediates of the Formula (XVIII) and Formula (XVI) includes the steps of cleavage of chiral epoxide compound of formula VII using suitable amine derivative to give compound of formula VIII (a); protection of compound of formula VIII (a) with suitable protecting agent to give compound of formula IX (a); condensation of compound of formula IX (a) with formula XVI in presence of base to give compound of formula XVII; and optionally using debenzylation or deprotection of formula XVII in presence of debenzylating agent or deprotecting agent.

The process of synthesis of Abediterol from intermediate (VII) via intermediates of the Formula (XX) and Formula (XVI) includes following steps of cleavage of chiral epoxide compound of formula VII using suitable amine derivative to give compound of formula VIII (a); protection of compound of formula VIII (a)with suitable protecting agent to give compound of formula IX (a); debenzylation of compound of formula IX (a) using debenzylating agent to give compound of formula XX; and condensation of compound of formula XX with formula XVI in presence of base.

In these processes, in the step of carbonylation, the solvent is selected from tetrahydrofuran, methyl tert-butyl ether, diisopropyl ether, or diethyl ether, the base is selected from n-butyl lithium, s-butyl lithium, lithium diisopropylamide, potassium bis(trimethylsilyl)amide, and the mixture of solvents is N, N-dimethylformamide and tetrahydrofuran in the ratio of 1:1; and carbonylation is carried out at the temperature of about −78° C. to 0⁰C. In the step of chiral epoxidation, the base is selected from potassium hydroxide, sodium hydroxide, lithium hydroxide, in tert-butyl alcohol, isopropyl alcohol, methanol. In the step of cleavage of chiral epoxide, the solvents are selected from Tetrahydrofuran, or halo solvents; and the brominating agent is selected from bromine, 1,3-dibromohydantoin, Tetra n-butyl ammonium tri bromide, Tetra n-butyl ammonium tri bromide, NBS; and the cleavage of chiral epoxide is carried out under cooling. In the step of condensation, the solvents are selected from acetone, THF or DMF, acetonitrile, 2-methyl THF, MIBK; and the base is selected from group of K₂CO₃, Na₂CO₃, NaOH, KOH or Cs₂CO₃. In the step of benzylation, the benzylating agent is selected from benzyl bromide, benzyl chloride, benzyl iodide; and the benzylating step is carried out at the temperature of about 0° C. to 25° C. In the step of debenzylation the debenzylating agents are selected from Pd/C, Pd/BaSO₄, or Raney nickel. In the step of protection, the protecting agents are selected from THP, TBDMS, TMS and benzyl bromide, benzyl chloride, benzyl iodide; the solvents are selected from acetone, THF, DMF, acetonitrile, 2-metyl THF, MIBK, halo solvents and the base is selected from K₂CO₃, Na₂CO₃, Cs₂CO₃, imidazole. In the step of deprotection, the deprotection is carried out under acidic medium wherein the acid is selected from aq.HCl or acetic acid.

### DETAILED DESCRIPTION OF THE INVENTION

The foregoing objects of the present invention are accomplished and the problems and shortcomings associated with the prior art, techniques and approaches are overcome by the present invention as described below in the preferred embodiments.

All materials used herein were commercially purchased as described herein or prepared from commercially purchased materials as described herein.
Although specific terms are used in the following description for sake of clarity, these terms are intended to refer only to particular structure of the invention selected for illustration in the drawings and are not intended to define or limit the scope of the invention.

References in the specification to “preferred embodiment” means that a particular feature, structure, characteristic, or function described in detail thereby omitting known constructions and functions for clear description of the present invention.

In one aspect, the present invention relates to a process for preparation of Abediterol having the formula I.

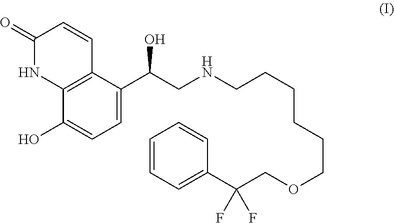 [SMILES: O=c1ccc2c([C@@H](O)CNCCCCCCOCC(F)(F)c3ccccc3)ccc(O)c2[nH]1]

In another aspect, the present invention relates to a process for the preparation of chiral intermediate compounds including compound of the Formula (VII).

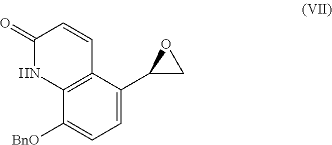 [SMILES: O=c1ccc2c([C@@H]3CO3)ccc(OCc3ccccc3)c2[nH]1]

In an embodiment, the preparation of the chiral intermediate compound of the Formula (VII) includes the steps of:
a) addition of the compound of Formula (II) to a solvent followed by adding a base and a benzylating agent to obtain a compound of Formula (III);

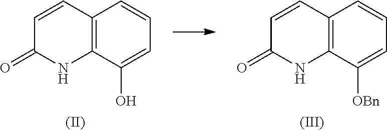 [SMILES: O=c1ccc2cccc(O)c2[nH]1 ; O=c1ccc2cccc(OCc3ccccc3)c2[nH]1 ; "(II)" ; "(III)"]

b) addition of the compound of Formula (III) to an acid and a brominating agent to make a reaction mixture; to obtain a compound of Formula (IV);

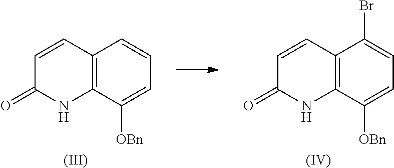 [SMILES: O=c1ccc2cccc(OCc3ccccc3)c2[nH]1 ; O=c1ccc2c(Br)ccc(OCc3ccccc3)c2[nH]1 ; "(III)" ; "(IV)"]

c) carbonylation of compound of formula IV in presence of a strong base to give compound of formula V;

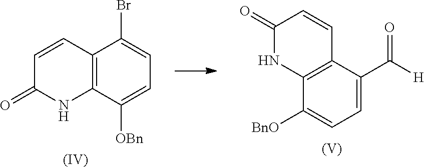

d) chiral epoxidation of compound of formula V using chiral sulphide of the formula (VI), in presence of base to give the compound of the formula VII;

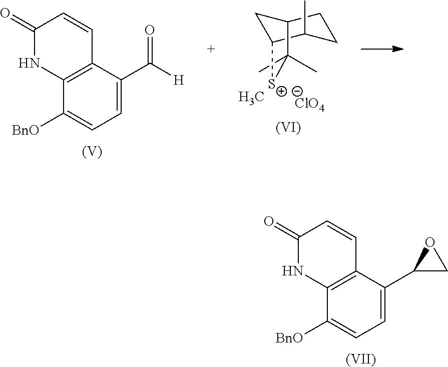

The detailed steps of the above-mentioned process are described herein:
In the step a), the compound of Formula (II) is 8-Hydroxyquinolin-2-(1H)-one. The solvent is selected from acetone, THF, DMF or like. The base is selected from potassium carbonate (K₂CO₃), Na₂CO₃, Cs₂CO₃ or like. The benzylating agent is selected from benzyl bromide, benzyl chloride, benzyl iodide, or like.
In the step b), the acid is selected from acetic acid, formic acid (HCO₂H), hydrobromic acid (HBr), or like. The brominating agent is selected from bromine, 1,3-dibromohydantoin, NBS or like.
In the step c), the carbonylation is carried out in presence of the solvents selected from tetrahydrofuran, methyl tert-butyl ether, diisopropyl ether, or diethyl ether; followed by treating with a strong base selected from n-butyl lithium, s-butyl lithium, lithium diisopropylamide, potassium bis(trimethylsilyl)amide; further followed by addition of a mixture of solvents, N, N-dimethylformamide and tetrahydrofuran in the ratio of 1:1 at the temperature of about −78° C. to 0⁰C;
In step d), the chiral epoxidation is carried out using the chiral sulphide derivative (VI), in presence of base selected from potassium hydroxide, sodium hydroxide, lithium hydroxide, in tert-butyl alcohol, isopropyl alcohol, methanol under cooling.

The compound Abediterol having the formula I is synthesized by various routes as described below:

The Route 1 of synthesis of compound of the Formula (I) from the intermediate of Formula (VII) includes the steps of:
a) cleavage of chiral epoxide compound of the formula VII using brominating agent to give a compound of the formula VIII;

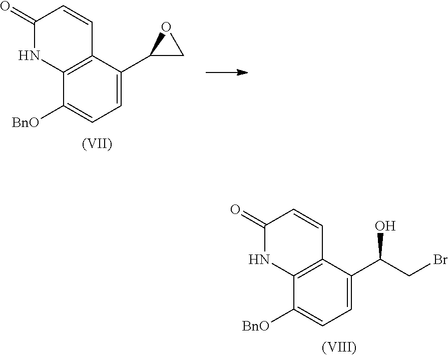

b) protection of compound of the formula VIII with a suitable protecting agent to give compound of the formula IX;

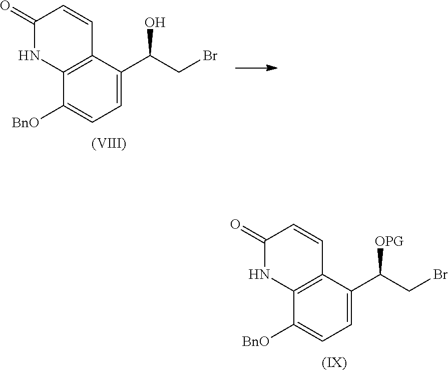

c) debenzylation of compound of the formula IX using debenzylating agent to give compound of the formula X;

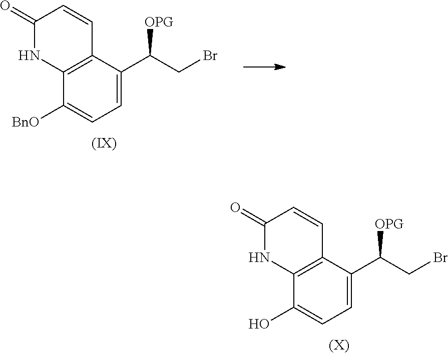

d) condensation of compound of the formula X with formula XI in presence of base to give compound of the formula XII; and

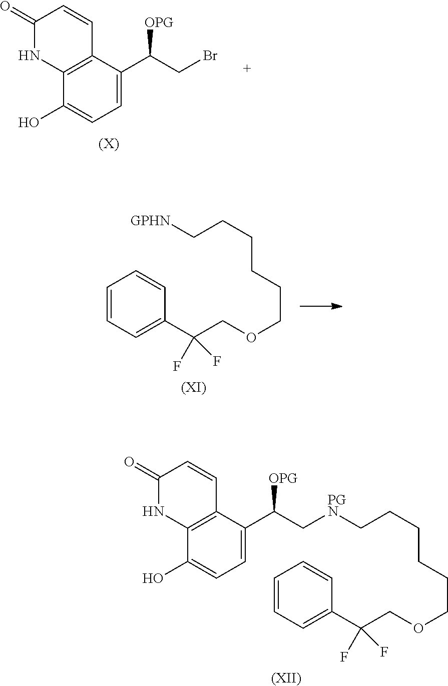

e) deprotection of compound of the formula XII using deprotecting agent to obtain compound of the Formula I.

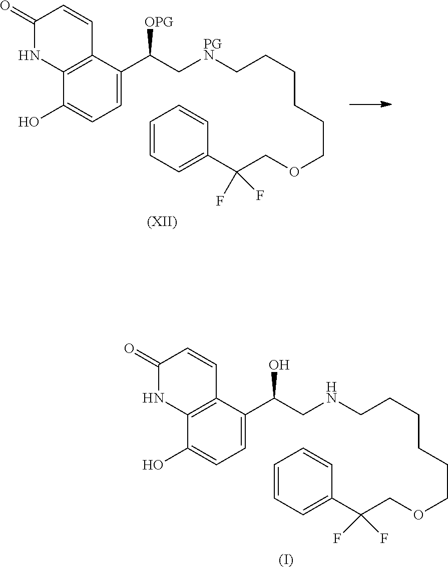

The Route 2 of synthesis of compound of the Formula (I) from the intermediate of Formula (VII) includes the steps of:
a) cleavage of chiral epoxide compound of the formula VII using brominating agent to give compound of the formula VIII;

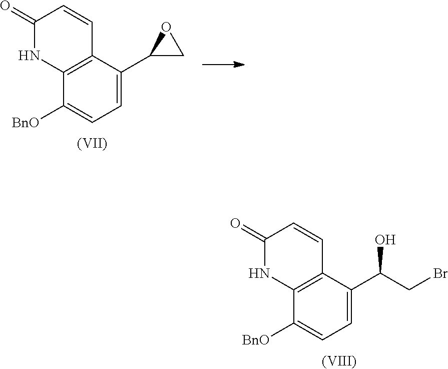

a) protection of compound of the formula VIII with suitable protecting agent to give compound of the formula IX;

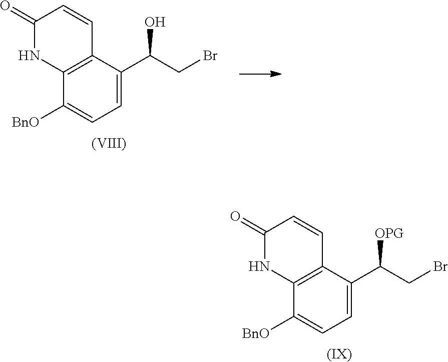

b) condensation of compound of the formula IX with formula XI in presence of base to give compound of the formula XIII; and

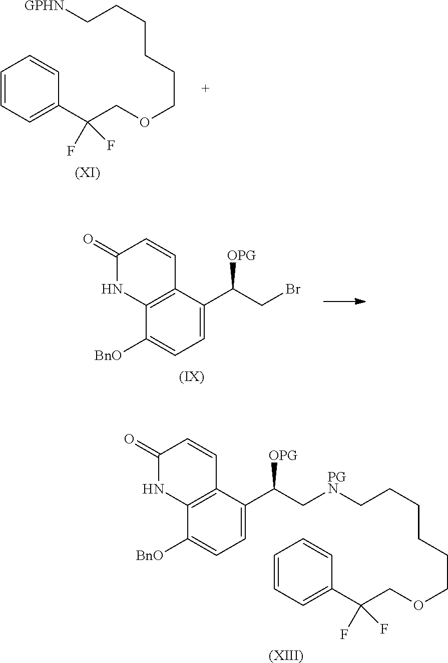

c) optionally using debenzylation or deprotection of formula XIII in presence of debenzylating agent or deprotecting agent to give compound of formula I.

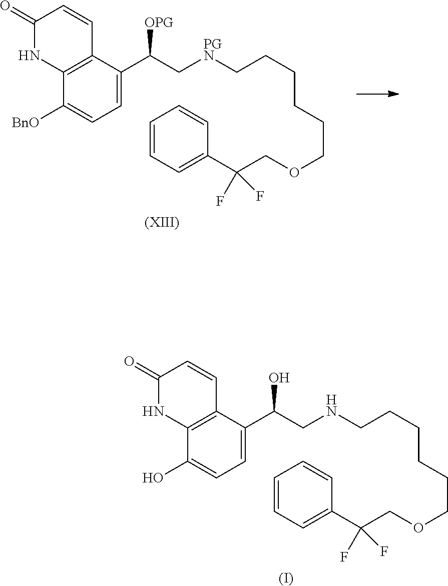

The Route 3 of synthesis of compound of the Formula (I) from the intermediate of Formula (VII) includes the steps of:
a) cleavage of chiral epoxide compound of formula VII using brominating agent to give compound of formula VIII;

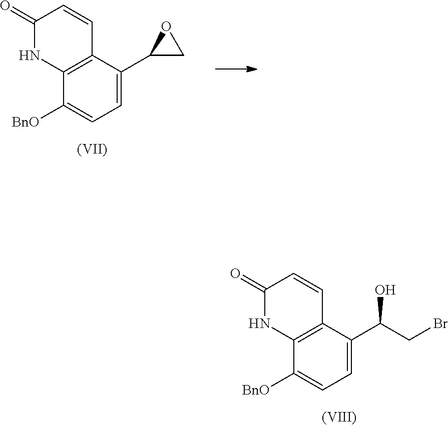

b) protection of compound of formula VIII with suitable protecting agent to give compound of formula IX;

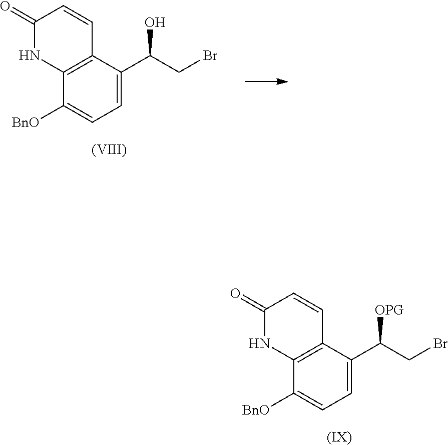

c) debenzylation of compound of formula IX using debenzylating agent to give compound of formula X;

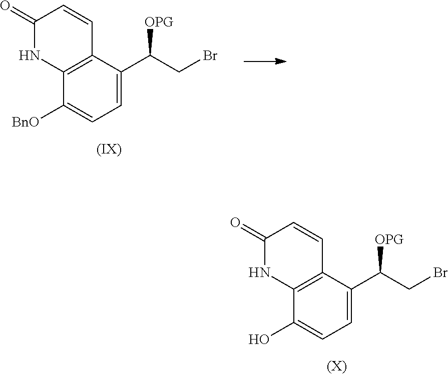

d) condensation of compound of formula X with formula XIV in presence of base to give compound of Formula I.

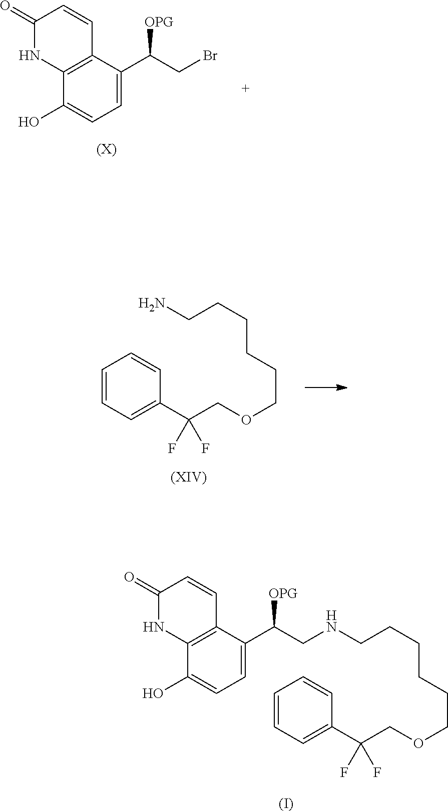

The Route 4 of synthesis of compound of the Formula (I) from the intermediate of Formula (VII) includes the steps of:
a) cleavage of chiral epoxide compound of formula VII using brominating agent to give compound of formula VIII;

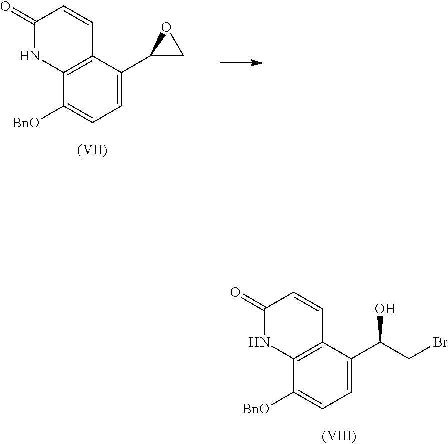

b) protection of compound of formula VIII with suitable protecting agent to give compound of formula IX;

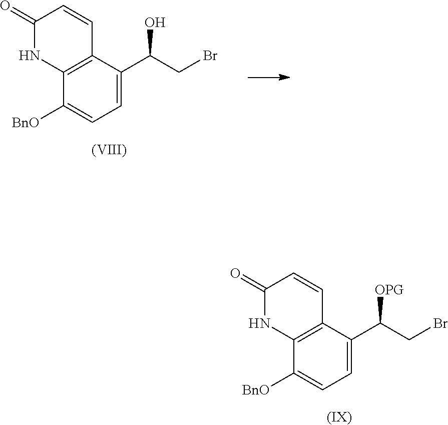

c) condensation of compound of formula IX with formula XIV in presence of base to give compound of formula XV;

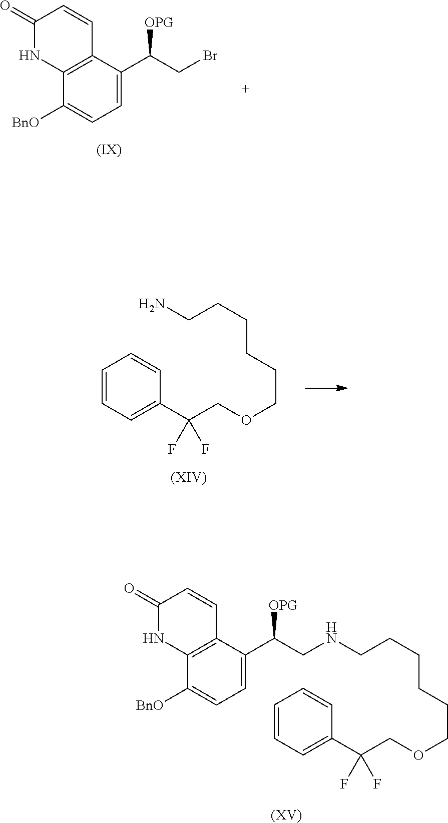

d) optionally using debenzylation or deprotection of compound of formula XV in presence of debenzylating agent or deprotecting agent to give Compound of Formula I.

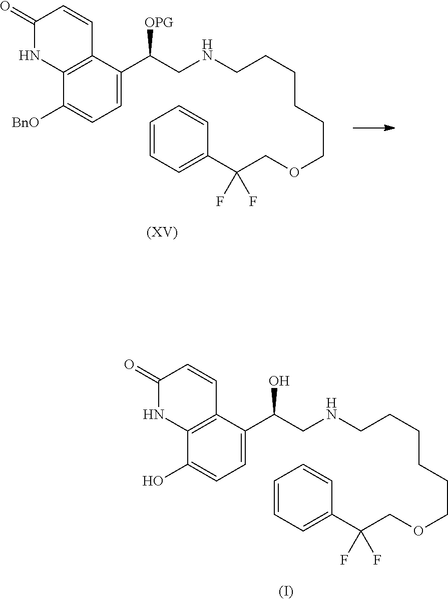

The Route 5 of synthesis of compound of the Formula (I) from the intermediate of Formula (VII) includes the steps of:
a) cleavage of chiral epoxide of formula VII using suitable amine derivative to give formula VIII (a);

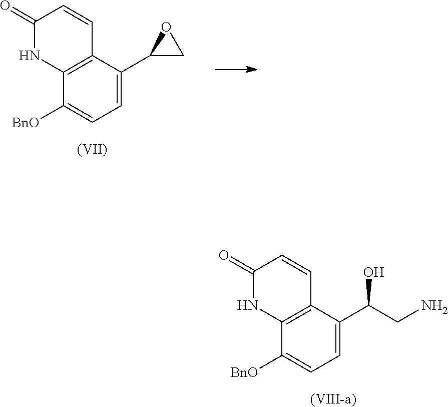

b) protection of compound of formula VIII (a) with suitable protecting agent to give compound of formula IX (a);

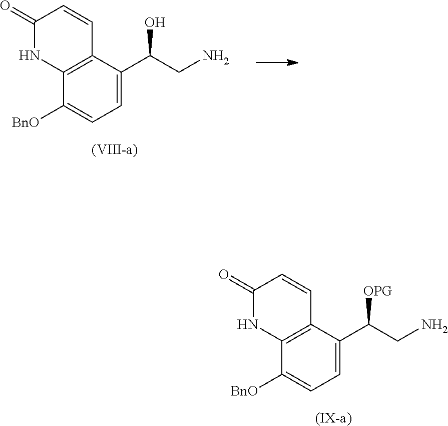

c) condensation of compound of formula IX (a) with formula XVI in presence of base to give compound of formula XVII; and

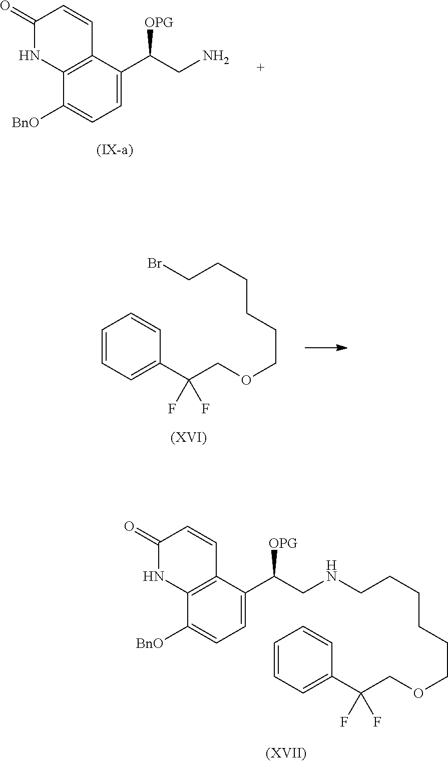

d) optionally using debenzylation or deprotection of formula XVII in presence of debenzylating agent or deprotecting agent.

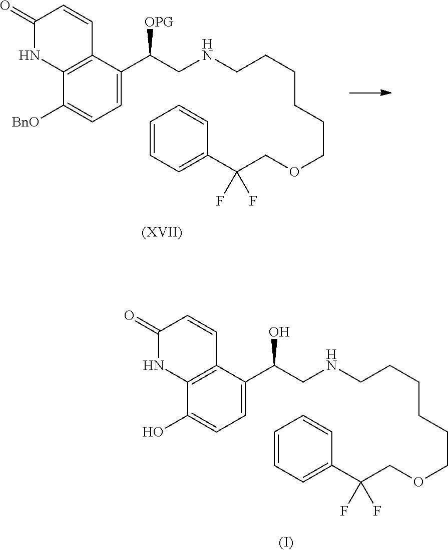

The Route 6 of synthesis of compound of the Formula (I) from the intermediate of Formula (VII) includes the steps of:
a) cleavage of chiral epoxide of compound of formula VII using aminating agent to give compound of formula VIII(a);

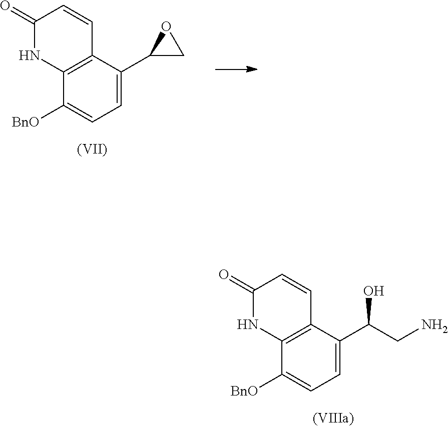

b) protection of compound of formula VIII (a) with suitable protecting agent to give compound of formula XVIII;

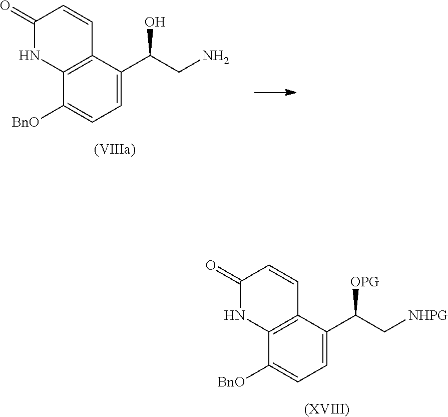

c) condensation of compound of formula XVIII with formula XVI in presence of base to give compound of formula XIX;

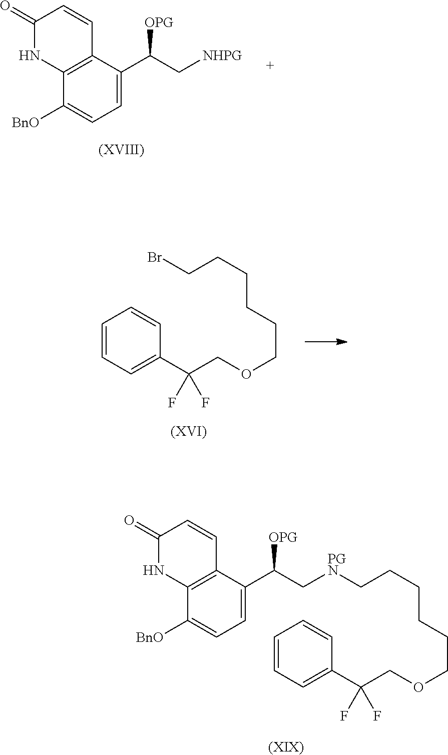

d) optionally using debenzylation or deprotection of formula XIX in presence of debenzylating agent or deprotecting agent.

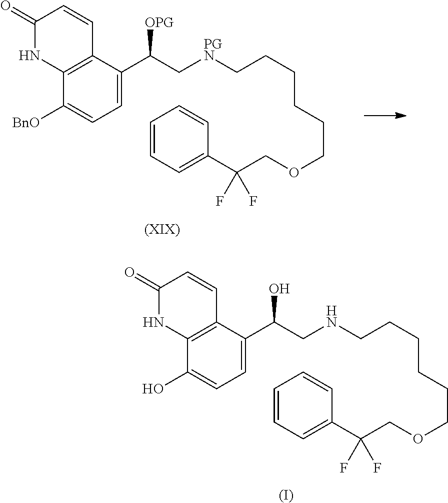

The Route 7 of synthesis of compound of the Formula (I) from the intermediate of Formula (VII) includes the steps of:
a) cleavage of chiral epoxide compound of formula VII using aminating agent to give compound of formula VIII(a);

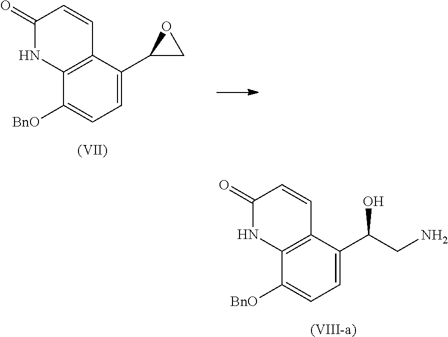

b) protection of compound of formula VIII (a) with suitable protecting agent to give compound of formula IX (a);

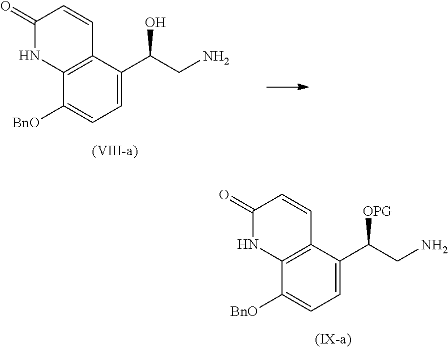

c) debenzylation of formula IX (a) using debenzylating agent to give compound of formula XX;

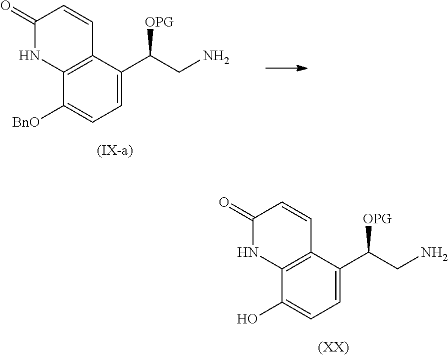

d) condensation of compound of formula XX with formula XVI in presence of base.

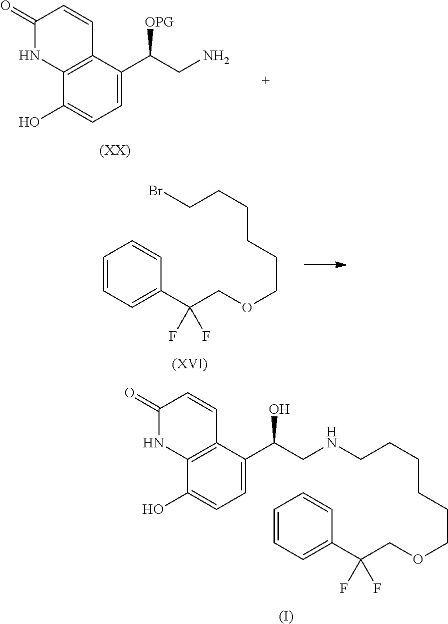

In all the above-mentioned routes for synthesis of the compound of the Formula (I) from the intermediate of Formula (VII), the detailed processes are described in detailed herein:
a) the cleavage of chiral epoxide is carried out in the solvents selected from Tetrahydrofuran, or 10 halo solvents; and the brominating agents selected from bromine, 1,3-dibromohydantoin, Tetra n-butyl ammonium tri bromide, Tetra n-butyl ammonium tri bromide, NBS; and the cleavage of chiral epoxide is carried out under cooling;
b) the condensation is carried out in presence of the solvents selected from acetone, THF or DMF, acetonitrile, 2-methyl THF, MIBK; and the base selected from group of K₂CO₃, Na₂CO₃, NaOH, KOH or Cs₂CO₃;
c) the benzylation is carried out in the presence of benzylating agents selected from benzyl bromide, benzyl chloride, benzyl iodide, at the temperature of about 0° C. to 25° C.;
d) the debenzylation is carried out in the presence of debenzylating agents selected from Pd/C, Pd/BaSO₄, or Raney nickel;
e) the protection is carried out in the presence of protecting agents selected from THP, TBDMS, TMS, benzyl bromide, benzyl chloride, benzyl iodide, in solvents selected from acetone, THF, DMF, acetonitrile, 2-metyl THF, MIBK, halo solvents using the base selected from K₂CO₃, Na₂CO₃, Cs₂CO₃, imidazole; and
f) the deprotection is carried out under acidic medium using acid selected from aq.HCl or by acetic acid.

These and other embodiments will be apparent to those of skill in the art and others in view of the following detailed description of some embodiments. It should be understood, however, that this summary, and the detailed description illustrate only some examples of various embodiments, and are not intended to be limiting to the invention as claimed.

The process of the present invention uses simple raw materials and reagents. Advantageously, the process of the present invention results in high yield of the end product with maximum purity.

The process of the present invention is cost effective. Further, the process is simple and uses safer process operations. It leads to reduction in the overall production time. Advantageously, the process does not use hypertoxic materials.

### EXAMPLES

Only a few examples and implementations are disclosed. Variations, modifications, and enhancements to the described examples and implementations and other implementations can be made based on what is disclosed.

Examples are set forth herein below and are illustrative of different amounts and types of reactants and reaction conditions that can be utilized in practicing the disclosure. It will be apparent, however, that the disclosure can be practiced with other amounts and types of reactants and reaction conditions than those used in the examples, and the resulting devices various different properties and uses in accordance with the disclosure above and as pointed out hereinafter.

### Example 1

### Preparation of Abediterol by Route 1 Synthesis From its Intermediates of the Formula (X) and Formula (XI):

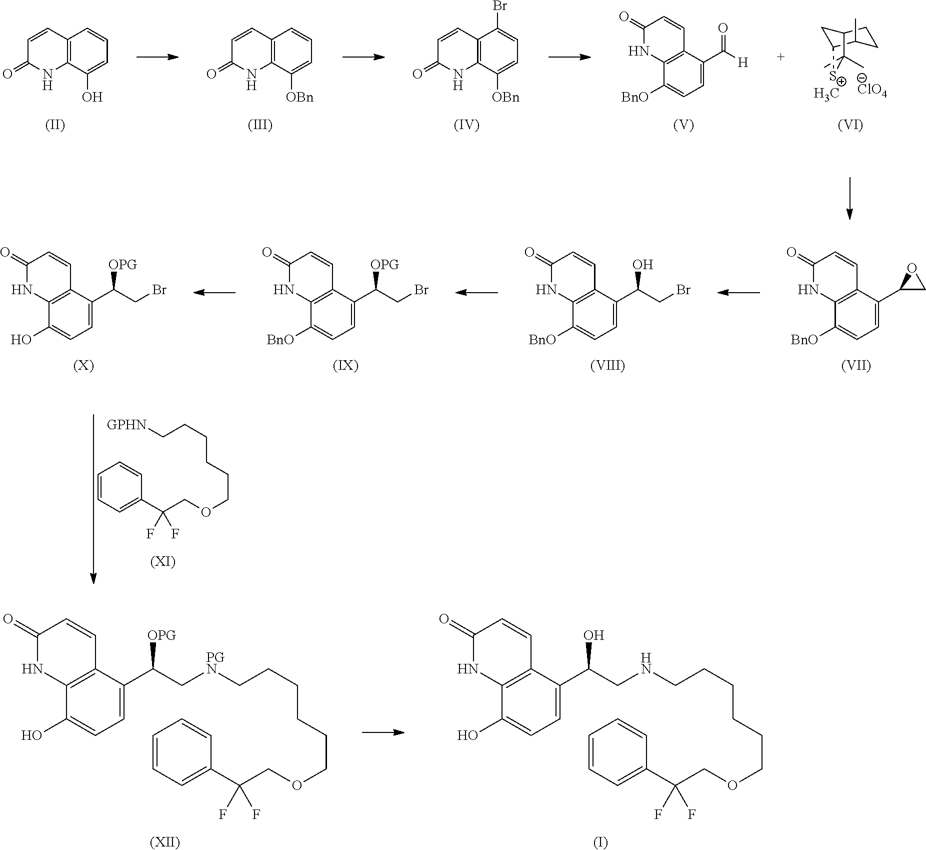 [0092] 1. To a solution of 8-Hydroxyquinolin-2-(1H)-one (II) (5.0 g, 0.031 mol) in acetone (40 mL), K₂CO₃ (5.1 g, 0.037 mol) and benzyl bromide (4.4 mL, 0.037 mol) were added at 0° C. The mixture was stirred at reflux temperature under nitrogen. After completion, the solvent was removed under reduced pressure and quenched with 1N HCl and extracted with ethyl acetate (3×50 mL), dried over sodium sulphate, filtered and distilled under reduced pressure. The residue was purified by silica gel chromatography to give the benzyl ether (III) as a colourless solid in 89% yield.
2. A solution of bromine (0.5 mL, 0.02 mol) in acetic acid (6.0 mL) was added dropwise to a solution of (III) (4.5 g, 0.018 mol) in acetic acid (40 mL). The mixture was stirred at room temperature for 4 h and quenched with sat. Na₂S2O₃ solution and then extracted with EtOAc (3×40 mL). The combined organic layers were washed with sat. NaHCO₃ solution followed by water and brine solution and dried over Na₂SO₄.
3. The bromo acetonide (IV) (3.7 g, 0.011 mol.) was dissolved in dry THF, cooled to −78° C. and a 1.6 M solution of n-butyl lithium (14.1 mL, 1.6 M in hexane, 0.022 mol.) was added drop wise. The mixture was stirred at −78° C. for 2 h and then treated with dry N, N-dimethylformamide (12.7 mL, 0.17 mol) as a 1:1 solution in dry THF. The resulting mixture was stirred at −78° C. for 0.75 h and warmed slowly up to room temperature. Then the mixture was diluted with diethyl ether, washed with water followed by a brine solution and dried over MgSO4. The solvent was removed in vacuo and the residue was purified by column chromatograph on silica gel to give the required compound (V) (74% yield).
4. A mixture of aldehyde (V) (2.5 g, 0.0089 mol), sulfonium perchlorate (VI) (3.2 g, 0.011 mol) and powdered KOH (0.62 g, 0.011 mol) in tert-butyl alcohol (40 mL) was stirred at room temperature for 48 h. Then the mixture was quenched with water and extracted with dichloromethane (30 mL×3). The combined extracts were washed with sat. NaCl and dried over MgSO₄. Evaporation of the solvent followed by purification on silica gel using a mixture of EtOAc—hexane as an eluent gave the epoxide (VII) in 80% yield.
5. A stirred solution of (VII) in THF was cooled to 10-15° C. and aq. HBr (48% 1.1 m. eq) was added. The resulting mixture was stirred at the same temperature till reaction completion. Then the reaction mixture was added to chilled water and extracted into ethyl acetate. The ethyl acetate layer was washed with water followed by saturated brine solution. The ethyl acetate layer was dried under anhydrous sodium sulphate. The ethyl acetate is distilled under vacuum at below 45° C. to get the compound bromo hydrin (VIII).
6. The compound (VIII) was protected using suitable protecting agent to give the protected product of formula (IX).
7. To the above compound (IX) (1.5 g, 0.0035 mol) in 30 ml of methanol was added 10% Pd/C (150 mg). The solution was placed in a stainless-steel reactor, which was then charged with hydrogen gas (150 psi). After being stirred for 24 h at room temperature, the mixture was filtered through Celite to remove the catalyst. The filtrate thus obtained was evaporated to give the product (X) (96% yield).
8. To a stirred solution of DMF (25 mL) and compound of formula (X) (5.0 g, 0.14 mol) a solution of compound (XI) (3.97 g, 0.015 mol) in DMF 25 ml was added in a dropwise manner at room temperature under nitrogen. After complete addition, the mixture was allowed to warm to 50° C. and stirred for 4 h. The resulting homogeneous mixture was quenched with saturated aqueous Na₂SO₃ and extracted with ethyl acetate. The ethyl acetate layer was washed with 10% aq. HCl solution, followed by water and saturated brine solution. The ethyl acetate layer was dried under anhydrous sodium sulphate at below 45° C. to get the compound (XII) (72% yield) as a pale-yellow residue.
9. To the above compound (XII) (3.5 g) in 30 ml of methanol was added 10% Pd/C (350 mg). The solution was placed in a stainless-steel reactor, 10% of Conc.HCl was added to the mass, which was then charged with hydrogen gas (150 psi). After being stirred for 24 h at room temperature, the mixture was filtered through Celite to remove the catalyst. The filtrate thus obtained was evaporated to give the product (I) (85% yield). Recrystallisation of (I) to give pure product with 75% yield.

### Example 2

### Preparation of Abediterol by Route 2 Synthesis From its Intermediates of the Formula (IX) and Formula (XI):

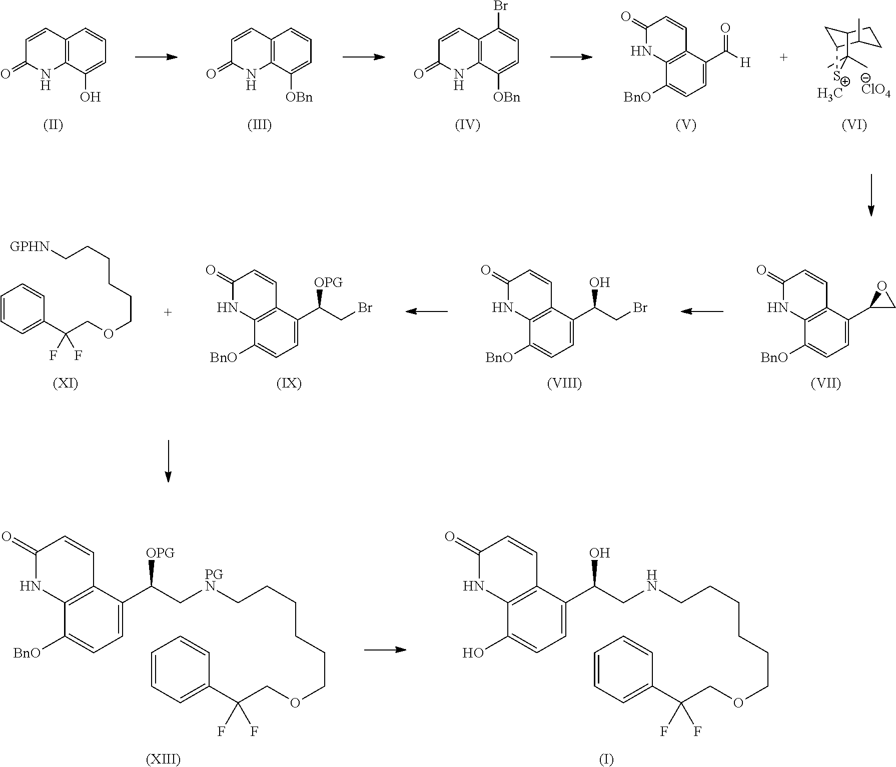 [0102] 1. To a solution of 8-Hydroxyquinolin-2-(1H)-one (II) (5.0 g, 0.031 mol) in acetone (40 mL), K₂CO₃ (5.1 g, 0.037 mol) and benzyl bromide (4.4 mL, 0.037 mol) were added at 0° C. The mixture was stirred at reflux temperature under nitrogen. After completion, the solvent was removed under reduced pressure and quenched with 1N HCl and extracted with ethyl acetate (3×50 mL), dried over sodium sulphate, filtered
   and distilled under reduced pressure. The residue was purified by silica gel chromatography to give the benzyl ether (III) as a colourless solid in 89% yield.
2. A solution of bromine (0.5 mL, 0.02 mol) in acetic acid (6.0 mL) was added dropwise to a solution of (III) (4.5 g, 0.018 mol) in acetic acid (40 mL). The mixture was stirred at room temperature for 4 h and quenched with sat. Na₂S2O₃ solution and then extracted with EtOAc (3×40 mL). The combined organic layers were washed with sat. NaHCO₃ solution followed by water and brine solution and dried over Na₂SO₄.
3 The bromo acetonide (IV) (3.7 g, 0.011 mol.) was dissolved in dry THF, cooled to −78° C. and a 1.6 M solution of n-butyl lithium (14.1 mL, 1.6 M in hexane, 0.022 mol.) was added drop wise. The mixture was stirred at −78° C. for 2 h and then treated with dry N, N-dimethylformamide (12.7 mL, 0.17 mol) as a 1:1 solution in dry THF. The resulting mixture was stirred at −78° C. for 0.75 h and warmed slowly up to room temperature. Then the mixture was diluted with diethyl ether, washed with water followed by a brine solution and dried over MgSO4. The solvent was removed in vacuo and the residue was purified by column chromatograph on silica gel to give the required compound (V) (74% yield).
4. A mixture of aldehyde (V) (2.5 g, 0.0089 mol), sulfonium perchlorate (VI) (3.2 g, 0.011 mol) and powdered KOH (0.62 g, 0.011 mol) in tert-butyl alcohol (40 mL) was stirred at room temperature for 48 h. Then the mixture was quenched with water and extracted with dichloromethane (30 mL×3). The combined extracts were washed with sat. NaCl and dried over MgSO₄. Evaporation of the solvent followed by purification on silica gel using a mixture of EtOAc—hexane as an eluent gave the epoxide (VII) in 80% yield.
5. A stirred solution of (VII) in THF was cooled to 10-15° C. and aq. HBr (48% 1.1 m. eq) was added. The resulting mixture was stirred at the same temperature till reaction completion. Then the reaction mixture was added to chilled water and extracted into ethyl acetate. The ethyl acetate layer was washed with water followed by saturated brine solution. The ethyl acetate layer was dried under anhydrous sodium sulphate. The ethyl acetate is distilled under vacuum at below 45° C. to get the compound bromo hydrin (VIII).
6. The compound (VIII) is protected using suitable protecting agent to give protected product of the formula (IX).
7. To a stirred solution of DMF (25 mL) and compound of formula (IX) (5.0 g) a solution of compound (XI) (3.97 g) in DMF 25 ml was added in a dropwise manner at room temperature under nitrogen. After complete addition, the mixture was allowed to warm to 50-60° C. and stirred for 4-6 h. The resulting homogeneous mixture was quenched with saturated aqueous Na₂SO₃ and extracted with ethyl acetate. The ethyl acetate layer was washed with 10% aq. HCl solution, followed by water and saturated brine solution. The ethyl acetate layer was dried under anhydrous sodium sulphate at below 45° C. to get the compound (XIII) (75% yield) as a pale-yellow residue.
8. To the above compound (XIII) (2.5 g) in 25 ml of methanol was added 10% Pd/C (250 mg). The solution was placed in a stainless-steel reactor, 10% of Conc.HCl was added to the mass, which was then charged with hydrogen gas (150 psi). After being stirred for 24 h at room temperature, the mixture was filtered through Celite to remove the catalyst. The filtrate thus obtained was evaporated to give the product (I) (83% yield). Recrystallisation of (I) to give pure product with 78% yield.

### Example 3

### Preparation of Abediterol by Route-3 Synthesis From its Intermediates of the Formula (X) and Formula (XIV):

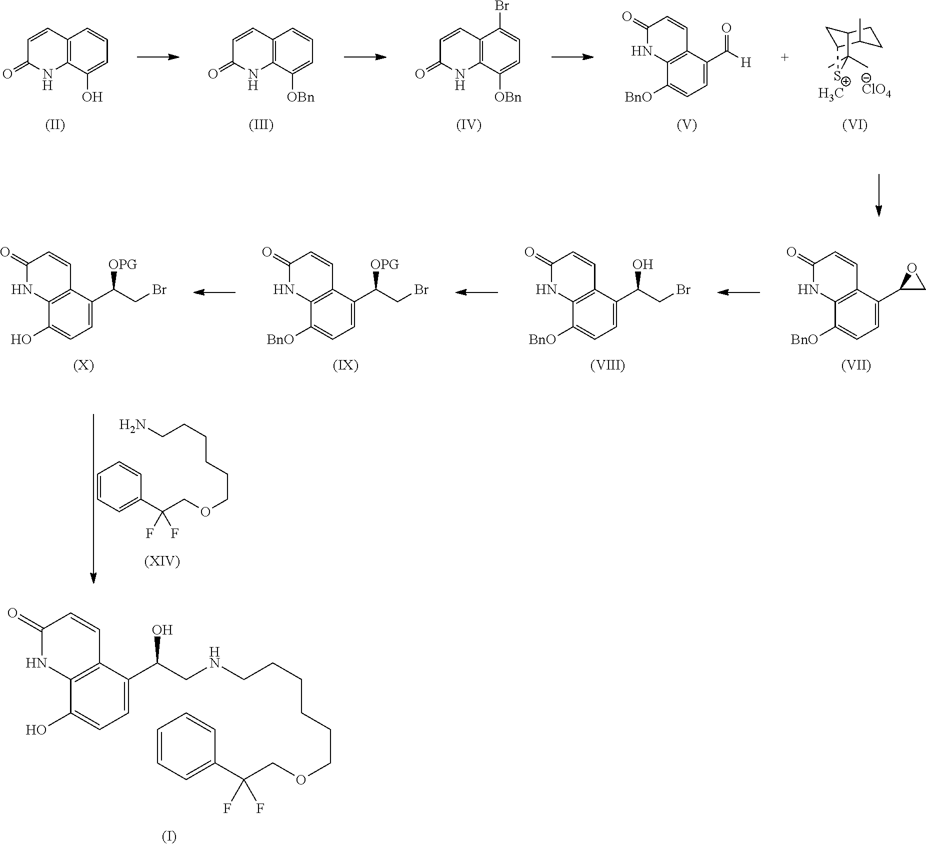 [0111] 1. To a solution of 8-Hydroxyquinolin-2-(1H)-one (II) (5.0 g, 0.031 mol) in acetone (40 mL), K₂CO₃ (5.1 g, 0.037 mol) and benzyl bromide (4.4 mL, 0.037 mol) were added at 0° C. The mixture was stirred at reflux temperature under nitrogen. After completion, the solvent was removed under reduced pressure and quenched with 1N HCl and extracted with ethyl acetate (3×50 mL), dried over sodium sulphate, filtered and distilled under reduced pressure. The residue was purified by silica gel chromatography to give the benzyl ether (III) as a colourless solid in 89% yield.
2. A solution of bromine (0.5 mL, 0.02 mol) in acetic acid (6.0 mL) was added dropwise to a solution of (III) (4.5 g, 0.018 mol) in acetic acid (40 mL). The mixture was stirred at room temperature for 4 h and quenched with sat. Na₂S2O₃ solution and then extracted with EtOAc (3×40 mL). The combined organic layers were washed with sat. NaHCO₃ solution followed by water and brine solution and dried over Na₂SO₄.
3. The bromo acetonide (IV) (3.7 g, 0.011 mol.) was dissolved in dry THF, cooled to −78° C. and a 1.6 M solution of n-butyl lithium (14.1 mL, 1.6 M in hexane, 0.022 mol.) was added drop wise. The mixture was stirred at −78° C. for 2 h and then treated with dry N, N-dimethylformamide (12.7 mL, 0.17 mol) as a 1:1 solution in dry THF. The resulting mixture was stirred at −78° C. for 0.75 h and warmed slowly up to room temperature. Then the mixture was diluted with diethyl ether, washed with water followed by a brine solution and dried over MgSO4. The solvent was removed in vacuo and the residue was purified by column chromatograph on silica gel to give the required compound (V) (74% yield).
4. A mixture of aldehyde (V) (2.5 g, 0.0089 mol), sulfonium perchlorate (VI) (3.2 g, 0.011 mol) and powdered KOH (0.62 g, 0.011 mol) in tert-butyl alcohol (40 mL) was stirred at room temperature for 48 h. Then the mixture was quenched with water and extracted with dichloromethane (30 mL×3). The combined extracts were washed with sat. NaCl and dried over MgSO₄. Evaporation of the solvent followed by purification on silica gel using a mixture of EtOAc—hexane as an eluent gave the epoxide (VII) in 80% yield.
5. A stirred solution of (VII) in THF was cooled to 10-15° C. and aq. HBr (48% 1.1 m. eq) was added. The resulting mixture was stirred at the same temperature till reaction completion. Then the reaction mixture was added to chilled water and extracted into ethyl acetate. The ethyl acetate layer was washed with water followed by saturated brine solution. The ethyl acetate layer was dried under anhydrous sodium sulphate. The ethyl acetate is distilled under vacuum at below 45° C. to get the compound bromo hydrin (VIII).
6. The compound (VIII) is protected using suitable protecting agent to give protected product of formula (IX).
7. To the above compound (IX) (1.5 g, 0.0035 mol) in 30 ml of methanol was added 10% Pd/C (150 mg). The solution was placed in a stainless-steel reactor, which was then charged with hydrogen gas (150 psi). After being stirred for 24 h at room temperature, the mixture was filtered through Celite to remove the catalyst. The filtrate thus obtained was evaporated to give the product (X) (96% yield).
8. To a stirred solution of DMF (25 mL), K₂CO₃ 1.5 eq. and compound of formula (X) (5.0 g) a solution of compound (XIV) (5.2 g) in DMF 25 ml was added in a dropwise manner at room temperature under nitrogen. After complete addition, the mixture was allowed to warm to 50-60° C. and stirred for 4-6 h. The resulting homogeneous mixture was quenched with saturated aqueous Na₂SO₃ and extracted with ethyl acetate. The ethyl acetate layer was washed with 10% aq. HCl solution, followed by water and saturated brine solution. The ethyl acetate layer was dried under anhydrous sodium sulphate at below 45° C. to get the compound (I) (75% yield). Recrystallisation of the compound gives product with 78% yield.

### Example 4

### Preparation of Abediterol by Route-4 Synthesis From its Intermediates of the Formula (IX) and Formula (XIV):

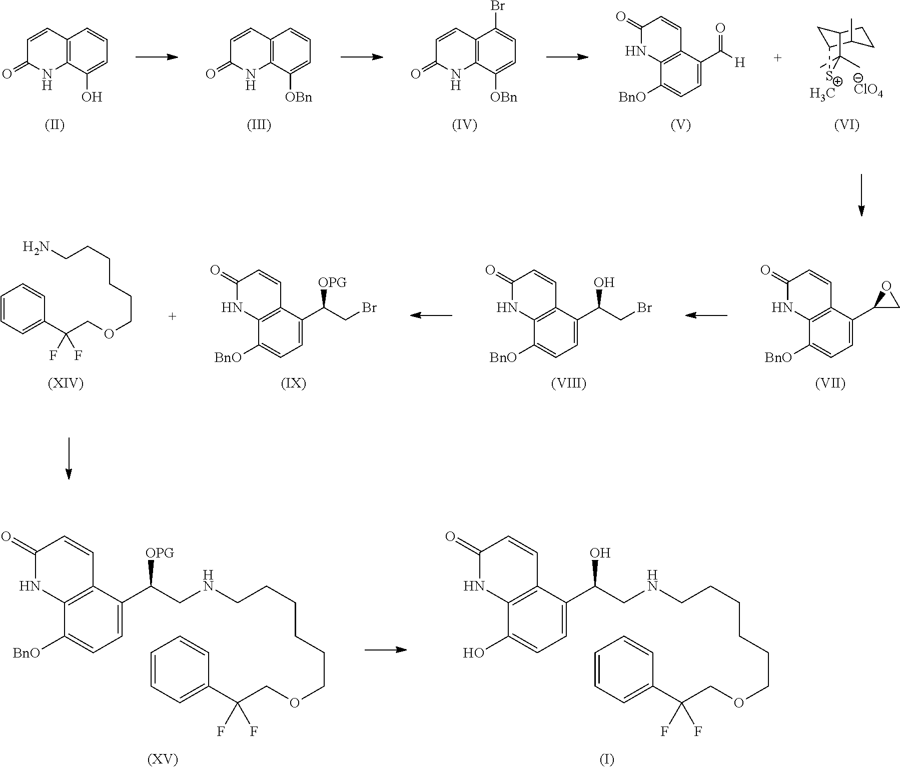 [0120] 1. To a solution of 8-Hydroxyquinolin-2-(1H)-one (II) (5.0 g, 0.031 mol) in acetone (40 mL), K₂CO₃ (5.1 g, 0.037 mol) and benzyl bromide (4.4 mL, 0.037 mol) were added at 0° C. The mixture was stirred at reflux temperature under nitrogen. After completion, the solvent was removed under reduced pressure and quenched with 1N HCl and extracted with ethyl acetate (3×50 mL), dried over sodium sulphate, filtered
   and distilled under reduced pressure. The residue was purified by silica gel chromatography to give the benzyl ether (III) as a colourless solid in 89% yield.
2. A solution of bromine (0.5 mL, 0.02 mol) in acetic acid (6.0 mL) was added dropwise to a solution of (III) (4.5 g, 0.018 mol) in acetic acid (40 mL). The mixture was stirred at room temperature for 4 h and quenched with sat. Na₂S2O₃ solution and then extracted with EtOAc (3×40 mL). The combined organic layers were washed with sat. NaHCO₃ solution followed by water and brine solution and dried over Na₂SO₄.
3. The bromo acetonide (IV) (3.7 g, 0.011 mol.) was dissolved in dry THF, cooled to −78° C. and a 1.6 M solution of n-butyl lithium (14.1 mL, 1.6 M in hexane, 0.022 mol.) was added drop wise. The mixture was stirred at −78° C. for 2 h and then treated with dry N, N-dimethylformamide (12.7 mL, 0.17 mol) as a 1:1 solution in dry THF. The resulting mixture was stirred at −78° C. for 0.75 h and warmed slowly up to room temperature. Then the mixture was diluted with diethyl ether, washed with water followed by a brine solution and dried over MgSO4. The solvent was removed in vacuo and the residue was purified by column chromatograph on silica gel to give the required compound (V) (74% yield).
4. A mixture of aldehyde (V) (2.5 g, 0.0089 mol), sulfonium perchlorate (VI) (3.2 g, 0.011 mol) and powdered KOH (0.62 g, 0.011 mol) in tert-butyl alcohol (40 mL) was stirred at room temperature for 48 h. Then the mixture was quenched with water and extracted with dichloromethane (30 mL×3). The combined extracts were washed with sat. NaCl and dried over MgSO4. Evaporation of the solvent followed by purification on silica gel using a mixture of EtOAc—hexane as an eluent gave the epoxide (VII) in 80% yield.
5. A stirred solution of (VII) in THF was cooled to 10-15° C. and aq. HBr (48% 1.1 m. eq) was added. The resulting mixture was stirred at the same temperature till reaction completion. Then the reaction mixture was added to chilled water and extracted into ethyl acetate. The ethyl acetate layer was washed with water followed by saturated brine solution. The ethyl acetate layer was dried under anhydrous sodium sulphate. The ethyl acetate is distilled under vacuum at below 45° C. to get the compound bromo hydrin (VIII).
6. The compound (VIII) is protected using suitable protecting agent to give protected product of the formula (IX).
7. To a stirred solution of DMF (25 mL), K₂CO₃ 1.5 eq. and compound of formula (X) (5.0 g) a solution of compound (XIV) (5.2 g) in DMF 25 ml was added in a dropwise manner at room temperature under nitrogen. After complete addition, the mixture was allowed to warm to 50-60° C. and stirred for 4-6 h. The resulting homogeneous mixture was quenched with saturated aqueous Na₂SO₃ and extracted with ethyl acetate. The ethyl acetate layer was washed with 10% aq. HCl solution, followed by water and saturated brine solution. The ethyl acetate layer was dried under anhydrous sodium sulphate at below 45° C. to get the compound (XV) (75% yield).
8. To the above compound (XV) (3.5 g) in 35 ml of methanol was added 10% Pd/C (350 mg). The solution was placed in a stainless-steel reactor, 10% of Conc.HCl was added to the mass, which was then charged with hydrogen gas (150 psi). After being stirred for 24 h at room temperature, the mixture was filtered through Celite to remove the catalyst. The filtrate thus obtained was evaporated to give the compound (I) (88% yield). Recrystallisation of compound (I) to give pure product with 80% yield.

### Example 5

### Preparation of Abediterol by Route-5 Synthesis From its Intermediates of the Formula (IXa) and Formula (XVI):

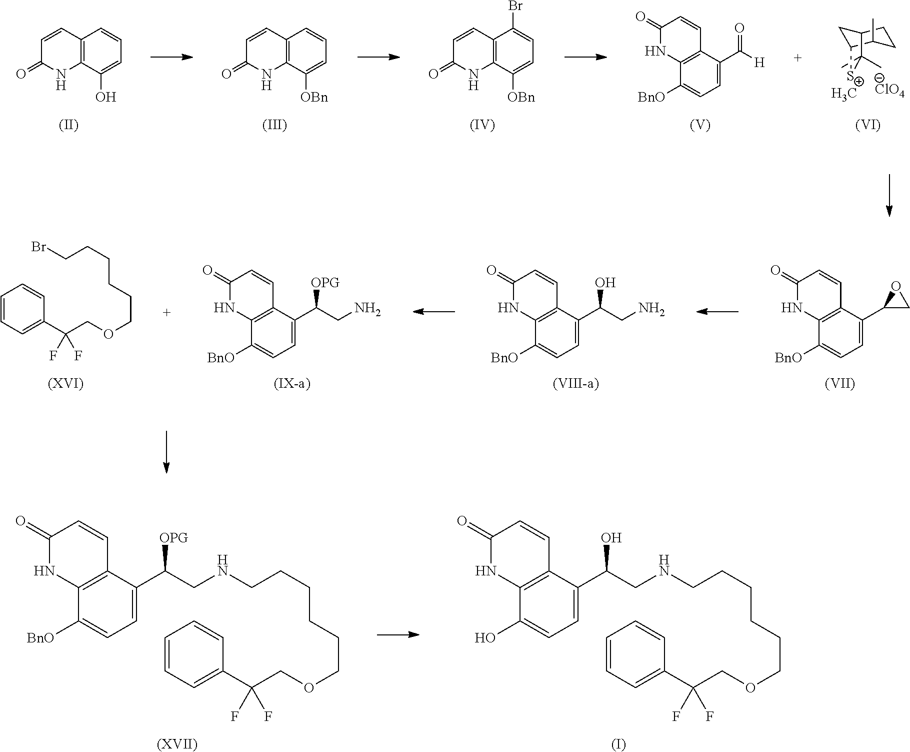 [0129] 1. To a solution of 8-Hydroxyquinolin-2-(1H)-one (II) (5.0 g, 0.031 mol) in acetone (40 mL), K₂CO₃ (5.1 g, 0.037 mol) and benzyl bromide (4.4 mL, 0.037 mol) were added at 0° C. The mixture was stirred at reflux temperature under nitrogen. After completion, the solvent was removed under reduced pressure and quenched with 1N HCl and extracted with ethyl acetate (3×50 mL), dried over sodium sulphate, filtered and distilled under reduced pressure. The residue was purified by silica gel chromatography to give the benzyl ether (III) as a colourless solid in 89% yield.
2. A solution of bromine (0.5 mL, 0.02 mol) in acetic acid (6.0 mL) was added dropwise to a solution of (III) (4.5 g, 0.018 mol) in acetic acid (40 mL). The mixture was stirred at room temperature for 4 h and quenched with sat. Na₂S2O₃ solution and then extracted with EtOAc (3×40 mL). The combined organic layers were washed with sat. NaHCO₃ solution followed by water and brine solution and dried over Na₂SO₄.
3. The bromo acetonide (IV) (3.7 g, 0.011 mol.) was dissolved in dry THF, cooled to −78° C. and a 1.6 M solution of n-butyl lithium (14.1 mL, 1.6 M in hexane, 0.022 mol.) was added drop wise. The mixture was stirred at −78° C. for 2 h and then treated with dry N, N-dimethylformamide (12.7 mL, 0.17 mol) as a 1:1 solution in dry THF. The resulting mixture was stirred at −78° C. for 0.75 h and warmed slowly up to room temperature. Then the mixture was diluted with diethyl ether, washed with water followed by a brine solution and dried over MgSO4. The solvent was removed in vacuo and the residue was purified by column chromatograph on silica gel to give the required compound (V) (74% yield).
4. A mixture of aldehyde (V) (2.5 g, 0.0089 mol), sulfonium perchlorate (VI) (3.2 g, 0.011 mol) and powdered KOH (0.62 g, 0.011 mol) in tert-butyl alcohol (40 mL) was stirred at room temperature for 48 h. Then the mixture was quenched with water and extracted with dichloromethane (30 mL×3). The combined extracts were washed with sat. NaCl and dried over MgSO₄. Evaporation of the solvent followed by purification on silica gel using a mixture of EtOAc—hexane as an eluent gave the epoxide (VII) in 80% yield.
5. A stirred solution of (VII) in THF was cooled to 10-15° C. and amine 1.1 m. eq was added. The resulting mixture was stirred at the room temperature till reaction completion. Then the reaction mixture was added to chilled water and extracted into ethyl acetate. The ethyl acetate layer was washed with water followed by saturated brine solution. The ethyl acetate layer was dried under anhydrous sodium sulphate. The ethyl acetate is distilled under vacuum at below 45° C. to get the compound (VIIIa).
6. The compound (VIIIa) is protected using suitable protecting agent to give protected product of the formula (IXa).
7. To a stirred solution of DMF (25 mL), K₂CO₃ 1.5 eq. and compound of formula (IXa) (5.0 g) a solution of compound (XVI) (5.5 g) in DMF 25 ml was added in a dropwise manner at room temperature under nitrogen. After complete addition, the mixture was allowed to warm to 50-60° C. and stirred for 4-6 h. The resulting homogeneous mixture was quenched with saturated aqueous Na₂SO₃ and extracted with ethyl acetate. The ethyl acetate layer was washed with 10% aq. HCl solution, followed by water and saturated brine solution. The ethyl acetate layer was dried under anhydrous sodium sulphate at below 45° C. to get the compound (XVII) (75% yield).
8. To the above compound (XVII) (2.5 g) in 25 ml of methanol was added 10% Pd/C (350 mg). The solution was placed in a stainless-steel reactor, 10% of Conc.HCl was added to the mass, which was then charged with hydrogen gas (150 psi). After being stirred for 24 h at room temperature, the mixture was filtered through Celite to remove the catalyst. The filtrate thus obtained was evaporated to give the compound (I) (85% yield). Recrystallisation of compound (I) to give pure product with 80% yield.

### Example 6

### Preparation of Abediterol by Route-6 Synthesis From its Intermediates of the Formula (XVIII) and Formula (XVI):

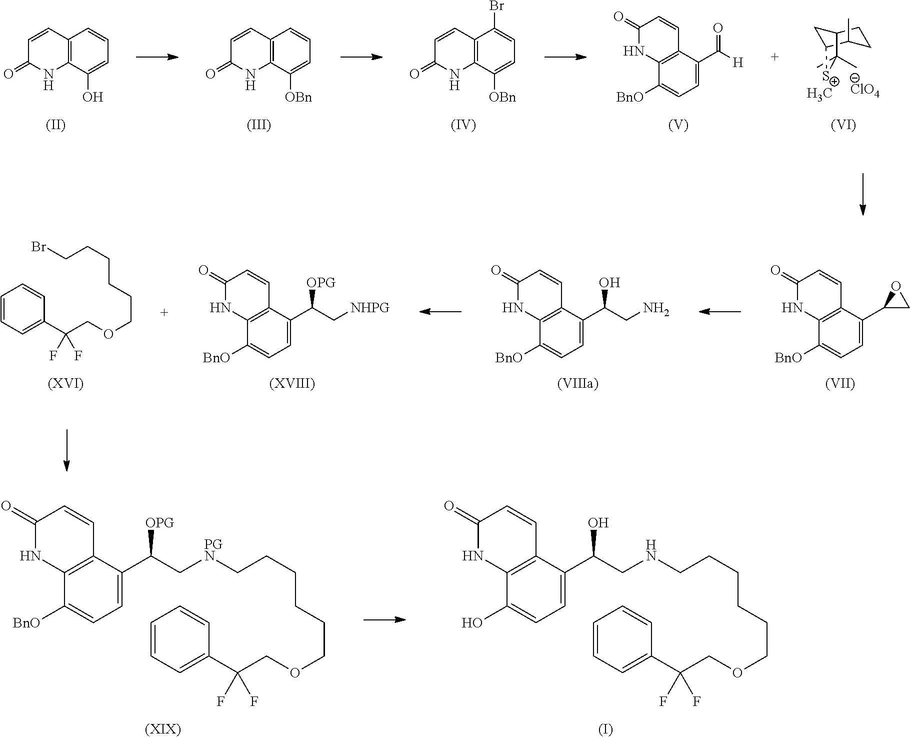 [0138] 1. To a solution of 8-Hydroxyquinolin-2-(1H)-one (II) (5.0 g, 0.031 mol) in acetone (40 mL), K₂CO₃ (5.1 g, 0.037 mol) and benzyl bromide (4.4 mL, 0.037 mol) were added at 0° C. The mixture was stirred at reflux temperature under nitrogen. After completion, the solvent was removed under reduced pressure and quenched with 1N HCl and extracted with ethyl acetate (3×50 mL), dried over sodium sulphate, filtered and distilled under reduced pressure. The residue was purified by silica gel chromatography to give the benzyl ether (III) as a colourless solid in 89% yield.
2. A solution of bromine (0.5 mL, 0.02 mol) in acetic acid (6.0 mL) was added dropwise to a solution of (III) (4.5 g, 0.018 mol) in acetic acid (40 mL). The mixture was stirred at room temperature for 4 h and quenched with sat. Na₂S2O₃ solution and then extracted with EtOAc (3×40 mL). The combined organic layers were washed with sat. NaHCO₃ solution followed by water and brine solution and dried over Na₂SO₄.
3. The bromo acetonide (IV) (3.7 g, 0.011 mol.) was dissolved in dry THF, cooled to −78° C. and a 1.6 M solution of n-butyl lithium (14.1 mL, 1.6 M in hexane, 0.022 mol.) was added drop wise. The mixture was stirred at −78° C. for 2 h and then treated with dry N, N-dimethylformamide (12.7 mL, 0.17 mol) as a 1:1 solution in dry THF. The resulting mixture was stirred at −78° C. for 0.75 h and warmed slowly up to room temperature. Then the mixture was diluted with diethyl ether, washed with water followed by a brine solution and dried over MgSO4. The solvent was removed in vacuo and the residue was purified by column chromatograph on silica gel to give the required compound (V) (74% yield).
4. A mixture of aldehyde (V) (2.5 g, 0.0089 mol), sulfonium perchlorate (VI) (3.2 g, 0.011 mol) and powdered KOH (0.62 g, 0.011 mol) in tert-butyl alcohol (40 mL) was stirred at room temperature for 48 h. Then the mixture was quenched with water and extracted with dichloromethane (30 mL×3). The combined extracts were washed with sat. NaCl and dried over MgSO₄. Evaporation of the solvent followed by purification on silica gel using a mixture of EtOAc—hexane as an eluent gave the epoxide (VII) in 80% yield.
5. A stirred solution of (VII) in THF was cooled to 10-15° C. and amine 1.1 m. eq was added. The resulting mixture was stirred at the room temperature till reaction completion. Then the reaction mixture was added to chilled water and extracted into ethyl acetate. The ethyl acetate layer was washed with water followed by saturated brine solution. The ethyl acetate layer was dried under anhydrous sodium sulphate. The ethyl acetate is distilled under vacuum at below 45° C. to get the compound (VIIIa).
6. The compound (VIIIa) is protected using suitable protecting agent to give protected product of the formula (XVIII).
7. To a stirred solution of DMF (25 mL), K₂CO₃ 1.5 eq. and compound of formula (XVIII) (5.0 g) a solution of compound (XVI) (5.5 g) in DMF 25 ml was added in a dropwise manner at room temperature under nitrogen. After complete addition, the mixture was allowed to warm to 50-60° C. and stirred for 4-6 h. The resulting homogeneous mixture was quenched with saturated aqueous Na₂SO₃ and extracted with ethyl acetate. The ethyl acetate layer was washed with 10% aq. HCl solution, followed by water and saturated brine solution. The ethyl acetate layer was dried under anhydrous sodium sulphate at below 45° C. to get the compound (XIX) (75% yield).
8. To the above compound (XIX) (2.5 g) in 25 ml of methanol was added 10% Pd/C (250 mg). The solution was placed in a stainless-steel reactor, 10% of Conc.HCl was added to the mass, which was then charged with hydrogen gas (150 psi). After being stirred for 24 h at room temperature, the mixture was filtered through Celite to remove the catalyst. The filtrate thus obtained was evaporated to give the compound (I) (85% yield). Recrystallisation of compound (I) to give pure product with 80% yield.

### Example 7

Preparation of Abediterol by Route-7 Synthesis From its intermediates of the Formula (XX) and Formula (XVI):

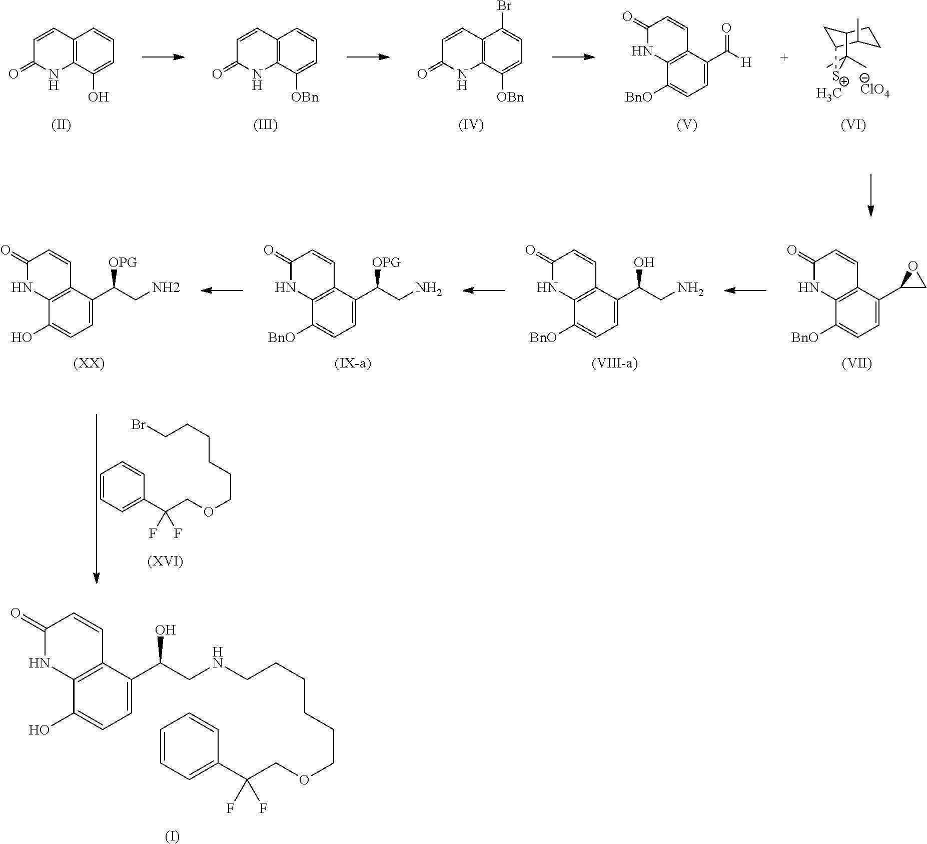

1. To a solution of 8-Hydroxyquinolin-2-(1H)-one (II) (5.0 g, 0.031 mol) in acetone (40 mL), K₂CO₃ (5.1 g, 0.037 mol) and benzyl bromide (4.4 mL, 0.037 mol) were added at 0° C. The mixture was stirred at reflux temperature under nitrogen. After completion, the solvent was removed under reduced pressure and quenched with 1N HCl and extracted with ethyl acetate (3×50 mL), dried over sodium sulphate, filtered and distilled under reduced pressure. The residue was purified by silica gel chromatography to give the benzyl ether (III) as a colourless solid in 89% yield.
2. A solution of bromine (0.5 mL, 0.02 mol) in acetic acid (6.0 mL) was added dropwise to a solution of (III) (4.5 g, 0.018 mol) in acetic acid (40 mL). The mixture was stirred at room temperature for 4 h and quenched with sat. Na₂S2O₃ solution and then extracted with EtOAc (3×40 mL). The combined organic layers were washed with sat. NaHCO₃ solution followed by water and brine solution and dried over Na₂SO₄.
3. The bromo acetonide (IV) (3.7 g, 0.011 mol.) was dissolved in dry THF, cooled to −78° C. and a 1.6 M solution of n-butyl lithium (14.1 mL, 1.6 M in hexane, 0.022 mol.) was added drop wise. The mixture was stirred at −78° C. for 2 h and then treated with dry N, N-dimethylformamide (12.7 mL, 0.17 mol) as a 1:1 solution in dry THF. The resulting mixture was stirred at −78° C. for 0.75 h and warmed slowly up to room temperature. Then the mixture was diluted with diethyl ether, washed with water followed by a brine solution and dried over MgSO4. The solvent was removed in vacuo and the residue was purified by column chromatograph on silica gel to give the required compound (V) (74% yield).
4. A mixture of aldehyde (V) (2.5 g, 0.0089 mol), sulfonium perchlorate (VI) (3.2 g, 0.011 mol) and powdered KOH (0.62 g, 0.011 mol) in tert-butyl alcohol (40 mL) was stirred at room temperature for 48 h. Then the mixture was quenched with water and extracted with dichloromethane (30 mL×3). The combined extracts were washed with sat. NaCl and dried over MgSO₄. Evaporation of the solvent followed by purification on silica gel using a mixture of EtOAc—hexane as an eluent gave the epoxide (VII) in 80% yield.
5. A stirred solution of (VII) in THF was cooled to 10-15° C. and amine 1.1 m. eq was added. The resulting mixture was stirred at the room temperature till reaction completion. Then the reaction mixture was added to chilled water and extracted into ethyl acetate. The ethyl acetate layer was washed with water followed by saturated brine solution. The ethyl acetate layer was dried under anhydrous sodium sulphate. The ethyl acetate is distilled under vacuum at below 45° C. to get the compound (VIIIa).
6. The compound(VIIIa) is protected using suitable protecting agent to give protected product of the formula (IXa).
7. To the above compound (IXa) (1.5 g) in 30 ml of methanol was added 10% Pd/C (150 mg). The solution was placed in a stainless-steel reactor, which was then charged with hydrogen gas (150 psi). After being stirred for 24 h at room temperature, the mixture was filtered through Celite to remove the catalyst. The filtrate thus obtained was evaporated to give the product (XX) (90% yield).
8. To a stirred solution of DMF (25 mL) and compound of formula (XX) (5.0 g) a solution of compound (XVI) (5.2 g) in DMF 25 ml was added in a dropwise manner at room temperature under nitrogen. After complete addition, the mixture was allowed to warm to 50-60° C. and stirred for 4-6 h. The resulting homogeneous mixture was quenched with saturated aqueous Na₂SO₃ and extracted with ethyl acetate. The ethyl acetate layer was washed with 10% aq. HCl solution, followed by water and saturated brine solution. The ethyl acetate layer was dried under anhydrous sodium sulphate at below 45° C. to get the compound (I) (85% yield). Recrystallisation of (I) to give pure product with 75% yield.

The embodiments were chosen and described in order to best explain the principles of the present invention and its practical application, to thereby enable others, skilled in the art to best utilize the present invention and various embodiments with various modifications as are suited to the particular use contemplated.

It is understood that various omission and substitutions of equivalents are contemplated as circumstance may suggest or render expedient, but such are intended to cover the application or implementation without departing from the spirit or scope of the present invention.

1. A process for preparation of Abediterol having the Formula I 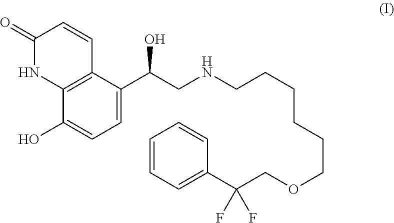 [SMILES: O=c1ccc2c([C@@H](O)CNCCCCCCOCC(F)(F)c3ccccc3)ccc(O)c2[nH]1]
or pharmaceutically acceptable salts, and the process for the preparation of the chiral intermediate compounds of Formula I wherein the process comprises the steps of:
a) addition of the compound of Formula (II) to a solvent followed by adding a base and a benzylating agent to obtain a compound of Formula (III); 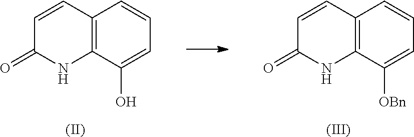 [SMILES: O=c1ccc2cccc(O)c2[nH]1 ; O=c1ccc2cccc(OCc3ccccc3)c2[nH]1 ; "(II)" ; "(III)"]
b) addition of the compound of Formula (III) to an acid and a brominating agent to make a reaction mixture; to obtain a compound of Formula (IV); 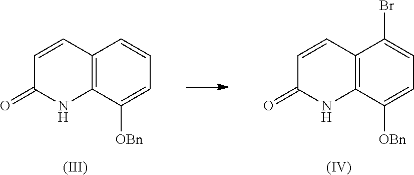 [SMILES: O=c1ccc2cccc(OCc3ccccc3)c2[nH]1 ; O=c1ccc2c(Br)ccc(OCc3ccccc3)c2[nH]1 ; "(III)" ; "(IV)"]
c) carbonylation of compound of formula IV with strong base to give compound of formula V; 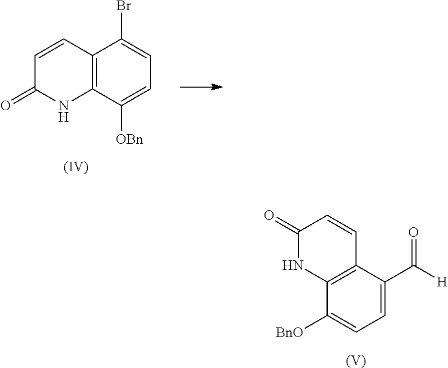
d) chiral epoxidation of compound of formula V using chiral sulphide (VI), in presence of base to give compound of formula VII; 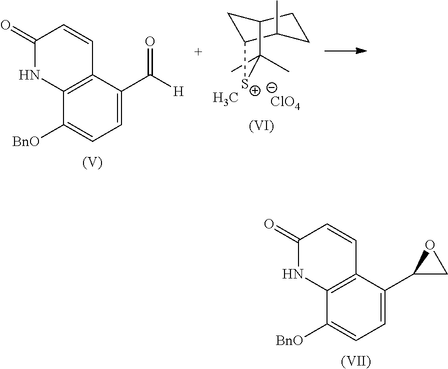
d) synthesis of Abediterol having the Formula (I) from the compound of Formula (VII)
(i) via intermediates of the Formula (X) and Formula (XI); or
(ii) via intermediates of the Formula (IX) and Formula (XI); or
(iii) via intermediates of the Formula (X) and Formula (XIV); or
(iv) via intermediates of the Formula (IX) and Formula (XIV); or
(v) via intermediates of the Formula (IXa) and Formula (XVI); or
(vi) via intermediates of the Formula (XVIII) and Formula (XVI); or
(vii) via intermediates of the Formula (XX) and Formula (XVI).

2. A process as claimed in claim 1, wherein synthesis of Abediterol from intermediate (VII) via intermediates of the Formula (X) and Formula (XI) includes the following steps:
a) cleavage of chiral epoxide of compound of formula VII using brominating agent to give compound of formula VIII; 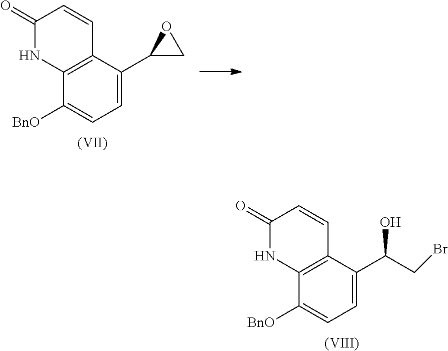
b) protection of compound of formula VIII with suitable protecting agent to give compound of formula IX; 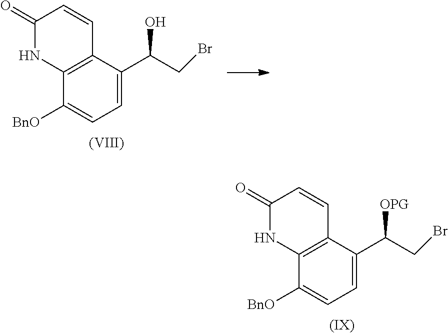
c) debenzylation of compound of formula IX using debenzylating agent to give compound of formula X; 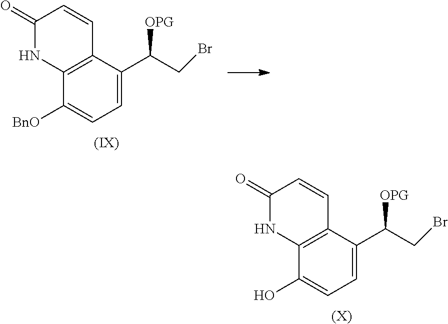
d) condensation of compound of formula X with formula XI in presence of base to give compound of formula XII; and 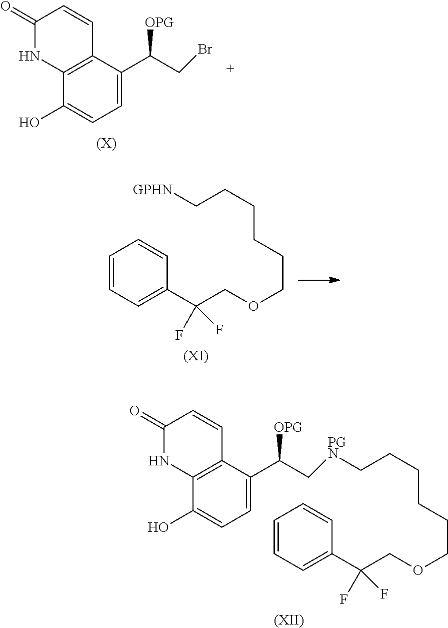
e) deprotection of compound of formula XII using deprotecting agent to give compound of the Formula I.

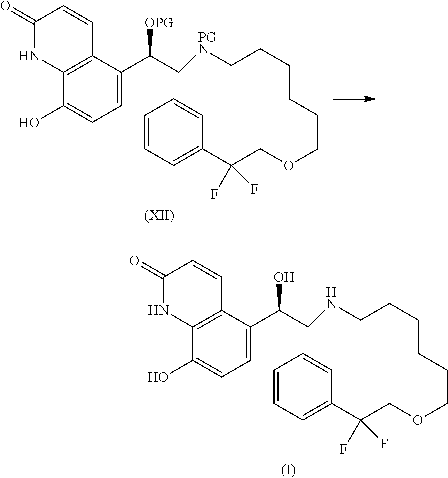

3. A process as claimed in claim 1, wherein synthesis of Abediterol from intermediate (VII) via intermediates of the Formula (IX) and Formula (XI) includes the following steps:
a) cleavage of chiral epoxide compound of formula VII using brominating agent to give compound of formula VIII; 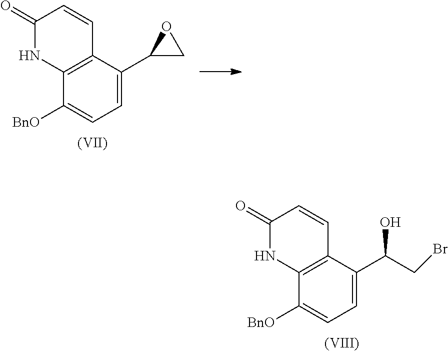
b) protection of compound of formula VIII with suitable protecting agent to give compound of formula IX; 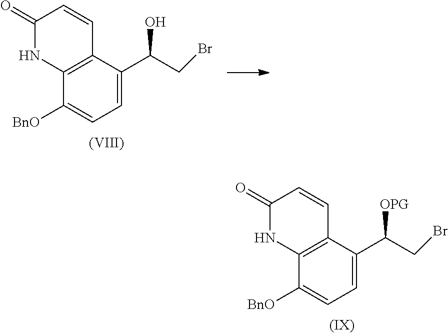
c) condensation of compound of formula IX with formula XI in presence of base to give compound of formula XIII; and 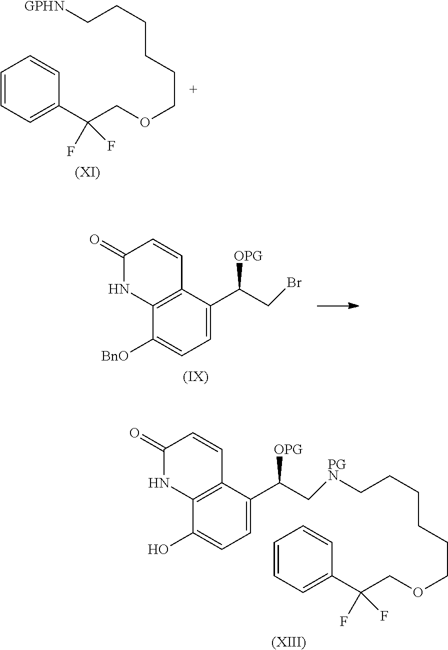
a) optionally using debenzylation or deprotection of compound of formula XIII in presence of debenzylating agent or deprotecting agent.

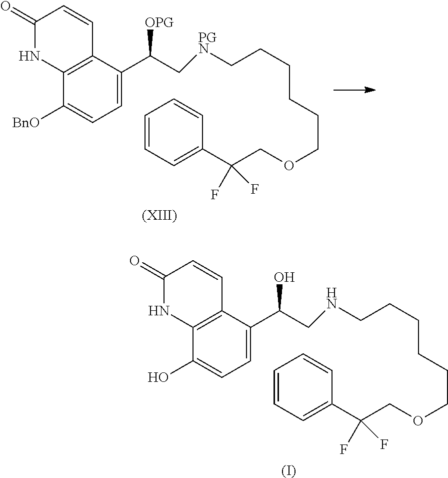

4. A process as claimed in claim 1, wherein synthesis of Abediterol from intermediate (VII) via intermediates of the Formula (X) and Formula (XIV) includes the following steps:
a) cleavage of chiral epoxide compound of formula VII using brominating agent to give compound of formula VIII; 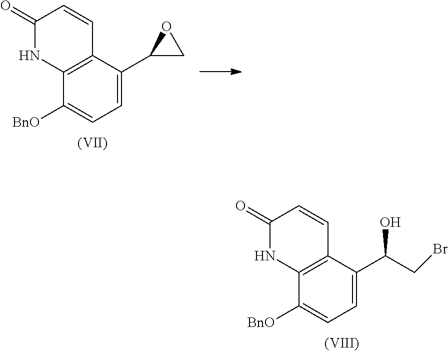
b) protection of compound of formula VIII with suitable protecting agent to give compound of formula IX; 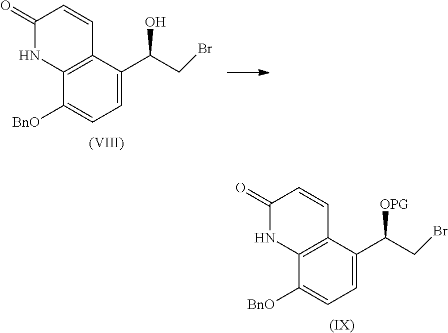
c) debenzylation of compound of formula IX using debenzylating agent to give compound of formula X; 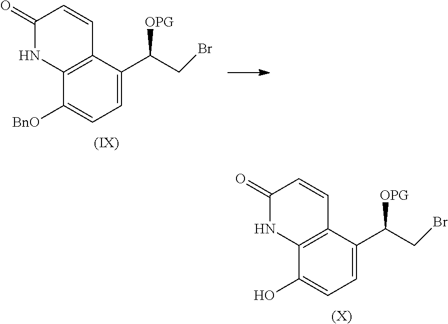
d) condensation of compound of formula X with formula XIV in presence of base.

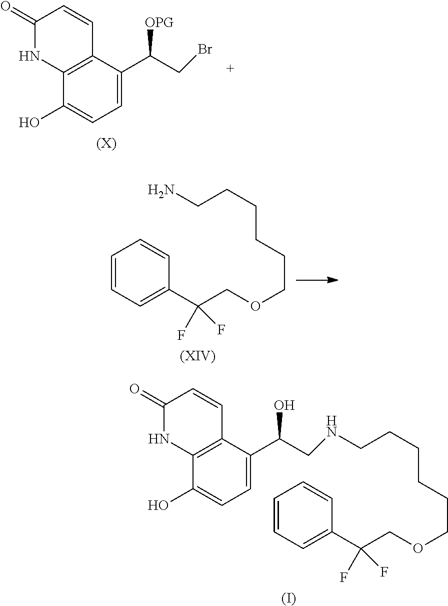

5. A process as claimed in claim 1, wherein synthesis of Abediterol from intermediate (VII) via intermediates of the Formula (IX) and Formula (XIV) includes the following steps:
a) cleavage of chiral epoxide compound of formula VII using brominating agent to give compound of formula VIII; 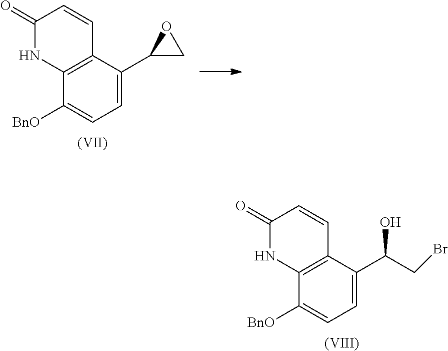
b) protection of compound of formula VIII with suitable protecting agent to give compound of formula IX; 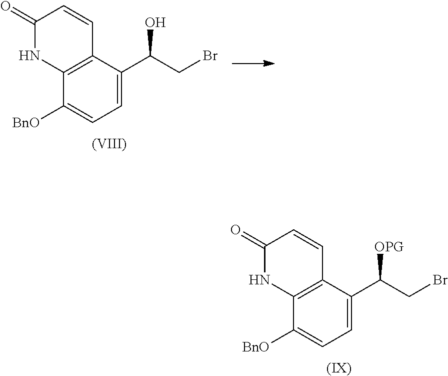
c) condensation of compound of formula IX with formula XIV in presence of base to give compound of formula XV; and 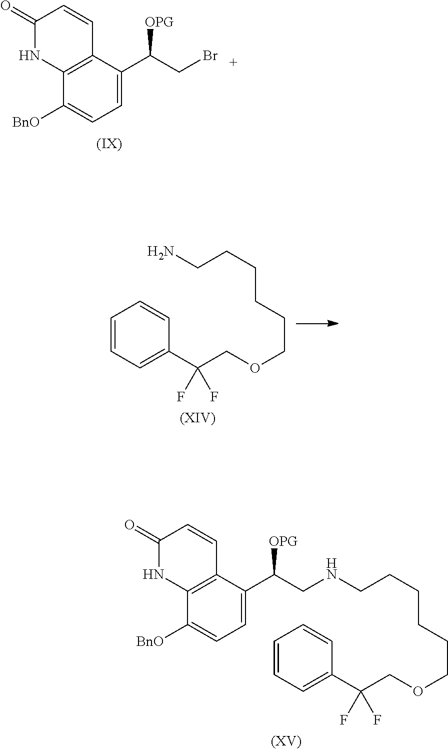
d) optionally using debenzylation or deprotection of formula XV in presence of debenzylating agent or deprotecting agent.

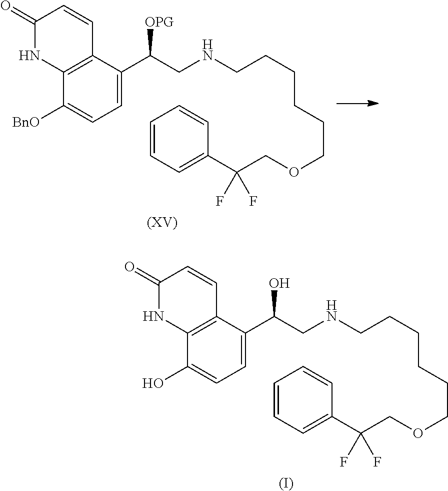

6. A process as claimed in claim 1, wherein synthesis of Abediterol from intermediate (VII) via intermediates of the Formula (IXa) and Formula (XVI);
includes the following steps:
a) cleavage of chiral epoxide compound of formula VII using suitable amine derivative to give compound of formula VIII (a); 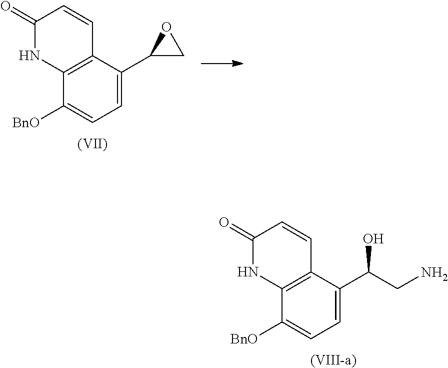
b) protection of compound of formula VIII (a) with suitable protecting agent to give compound of formula IX (a); 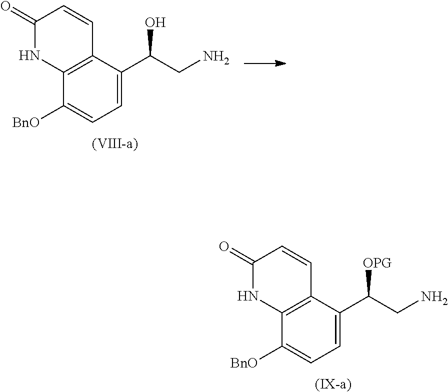
c) condensation of compound of formula IX (a) with formula XVI in presence of base to give compound of formula XVII; and 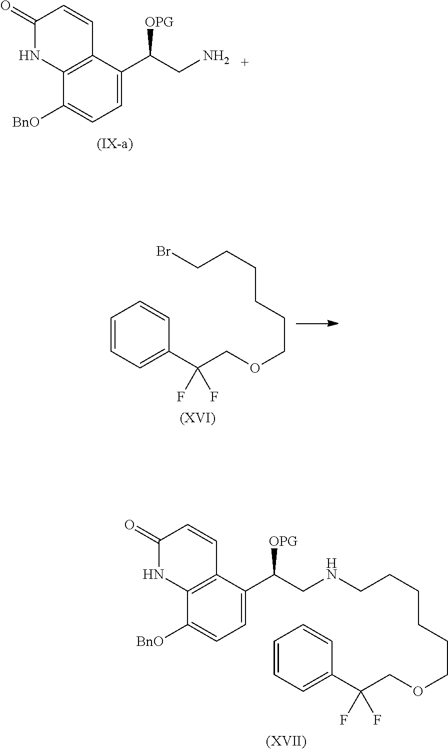
d) optionally using debenzylation or deprotection of formula XVII in presence of debenzylating agent or deprotecting agent.

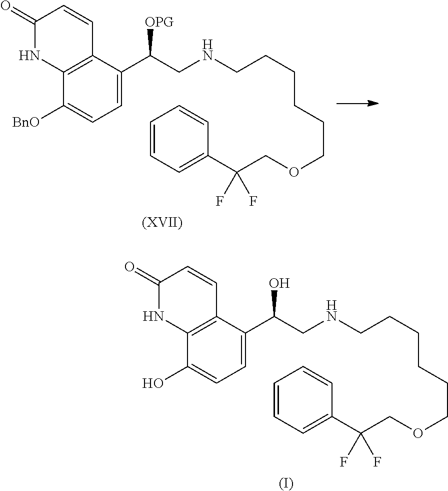

7. A process as claimed in claim 1, wherein synthesis of Abediterol from intermediate (VII) via intermediates of the Formula (XVIII) and Formula (XVI) includes the following steps:
a) cleavage of chiral epoxide compound of formula VII using suitable amine derivative to give compound of formula VIII (a); 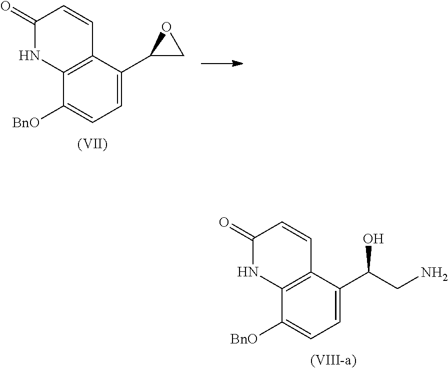
b) protection of compound of formula VIII (a) with suitable protecting agent to give compound of formula XVIII; 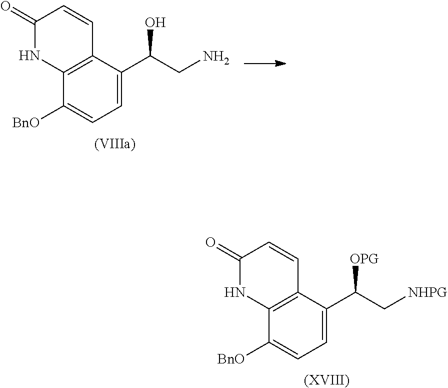
c) condensation of compound of formula XVIII with formula XVI in presence of base to give compound of formula XIX; and 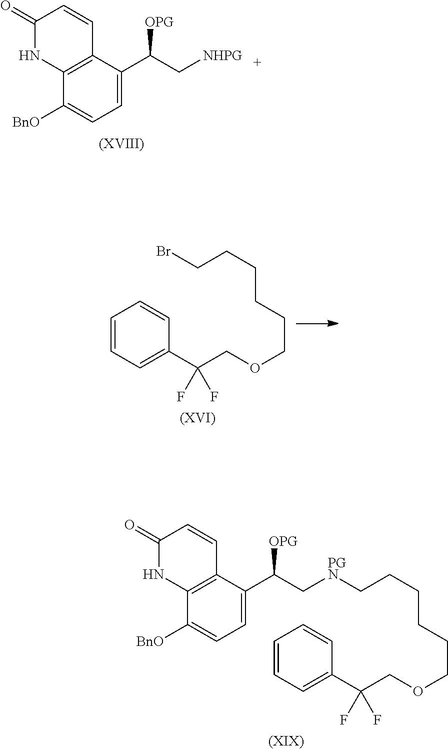
d) optionally using debenzylation or deprotection of formula XIX in presence of debenzylating agent or deprotecting agent.

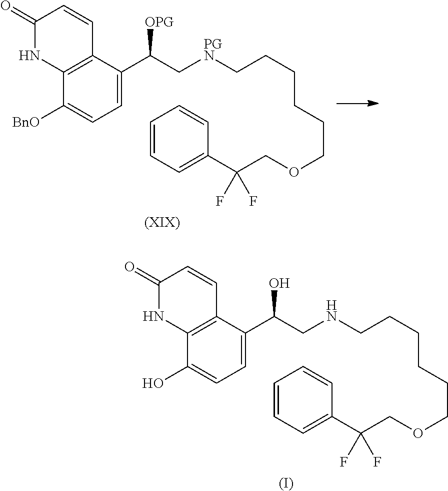

8. A process as claimed in claim 1, wherein synthesis of Abediterol from intermediate (VII) via intermediates of the Formula (XX) and Formula (XVI) includes the following steps:
a) cleavage of chiral epoxide compound of formula VII using suitable amine derivative to give compound of formula VIII (a); 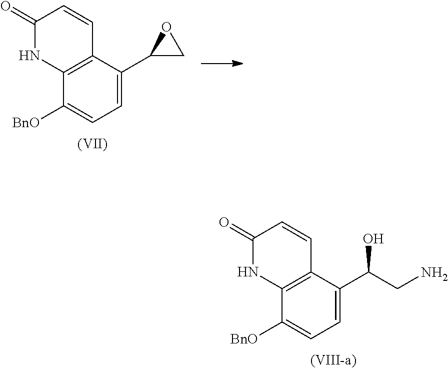
b) protection of compound of formula VIII (a)with suitable protecting agent to give compound of formula IX (a); 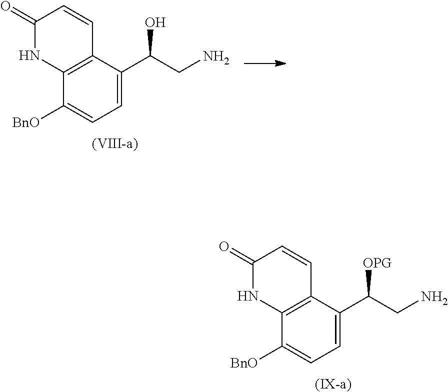
c) debenzylation of compound of formula IX (a) using debenzylating agent to give compound of formula XX; 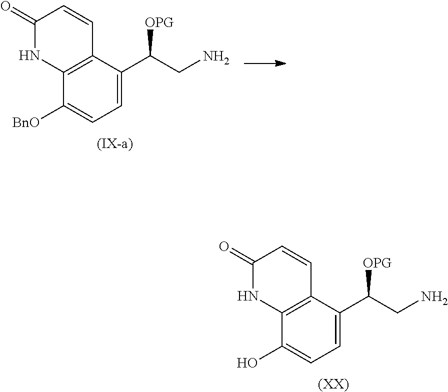
d) condensation of compound of formula XX with formula XVI in presence of base.

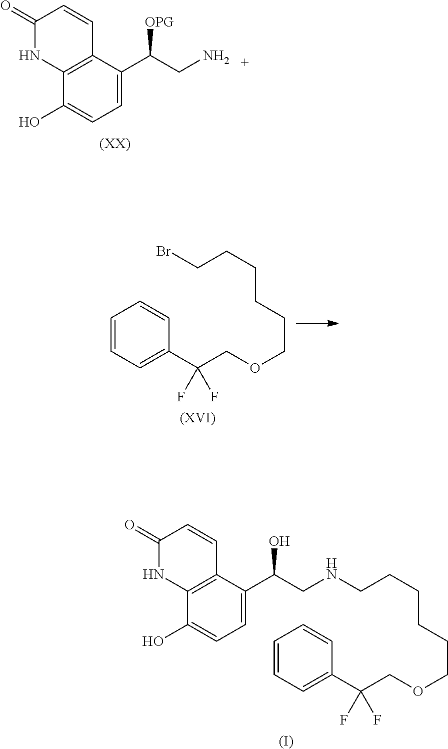

9. The process as claimed in claim 1, wherein in the step of carbonylation, the solvent is selected from tetrahydrofuran, methyl tert-butyl ether, diisopropyl ether, or diethyl ether, the base is selected from n-butyl lithium, s-butyl lithium, lithium diisopropylamide, potassium bis(trimethylsilyl)amide, and the mixture of solvents is N, N-dimethylformamide and tetrahydrofuran in the ratio of 1:1; and carbonylation is carried out at the temperature of about −78° C. to 0⁰C.

10. The process as claimed in claim 1, wherein in the step of chiral epoxidation the base is selected from potassium hydroxide, sodium hydroxide, lithium hydroxide, in tert-butyl alcohol, isopropyl alcohol, methanol.

11. The process as claimed in claims 2, wherein in the step of cleavage of chiral epoxide, the solvents are selected from Tetrahydrofuran, or halo solvents; and the brominating agent is selected from bromine, 1,3-dibromohydantoin, Tetra n-butyl ammonium tri bromide, Tetra n-butyl ammonium tri bromide, NB S; and the cleavage of chiral epoxide is carried out under cooling.

12. The process as claimed in claim 2, wherein in the step of condensation, the solvents are selected from acetone, THF or DMF, acetonitrile, 2-methyl THF, MIBK; and the base is selected from group of K₂CO₃, Na₂CO₃, NaOH, KOH or Cs₂CO₃.

13. The process as claimed in claim 2, wherein in the step of benzylation, the benzylating agent is selected from benzyl bromide, benzyl chloride, benzyl iodide; and the benzylating step is carried out at the temperature of about 0° C. to 25° C.

14. The process as claimed in claims 2, wherein in the step of debenzylation the debenzylating agents are selected from Pd/C, Pd/BaSO₄, or Raney nickel.

15. The process as claimed in claims 2, wherein in the step of protection, the protecting agents are selected from THP, TBDMS, TMS and benzyl bromide, benzyl chloride, benzyl iodide; the solvents are selected from acetone, THF, DMF, acetonitrile, 2-metyl THF, MIBK, halo solvents and the base is selected from K₂CO₃, Na₂CO₃, Cs₂CO₃, imidazole.

16. The process as claimed in claim 2, wherein in the step of deprotection, the deprotection is carried out under acidic medium wherein the acid is selected from aq.HCl or acetic acid.
